(19) United States
(12) Patent Application Publication
(10) Pub. No.: US 2011/0281301 A1
(43) Pub. Date: Nov. 17, 2011

(54) THE SECRETORY CAPACITY IN HOST CELLS

(75) Inventors: Hitto Kaufmann, Ulm (DE); Eric Becker, Hochdorf (DE); Lore Florin, Biberach (DE); Barbara Enenkel, Warthausen (DE); Kerstin Sautter, Biberach (DE); Rebecca Bischoff, Warthausen (DE)

(73) Assignee: BOEHRINGER INGELHEIM PHARMA GMBH & CO. KG, Ingelheim am Rhein (DE)

(21) Appl. No.: 12/742,299

(22) PCT Filed: Oct. 6, 2008
(86) PCT No.: PCT/EP2008/063308

§ 371 Date: Jul. 28, 2010

(30) Foreign Application Priority Data
Nov. 13, 2007 (EP) 07120563.7

(51) Int. Cl. C12P 21/00; C12N 5/10; C12N 15/85

(52) U.S. Cl. 435/69.6; 435/69.1; 435/455; 435/358

(57) ABSTRACT

The invention concerns the field of protein production and cell culture technology. It describes a method of producing a heterologous protein of interest in a cell comprising a. Increasing the expression or activity of a secretion enhancing gene, and b. Increasing the expression or activity of an anti-apoptotic gene, and c. Effecting the expression of said protein of interest, whereby the secretion enhancing gene is a gene encoding a protein whose expression or activity is induced during one of the following cellular processes: plasma-cell differentiation, unfolded protein response (UPR), endoplasmic reticulum overload response (EOR).

### BACKGROUND OF THE INVENTION

1. Technical Field

The invention concerns the field of cell culture technology. It concerns a method for producing proteins as well as host cells for biopharmaceutical manufacturing.

2. Background

The market for biopharmaceuticals for use in human therapy continues to grow at a high rate with 270 new biopharmaceuticals being evaluated in clinical studies and estimated sales of 30 billions in 2003 Biopharmaceuticals can be produced from various host cell systems, including bacterial cells, yeast cells, insect cells, plant cells and mammalian cells including human-derived cell lines. Currently, an increasing number of biopharmaceuticals is produced from eukaryotic cells due to their ability to correctly process and modify human proteins. Successful and high yield production of biopharmaceuticals from these cells is thus crucial and depends highly on the characteristics of the recombinant monoclonal cell line used in the process. Therefore, there is an urgent need to generate new host cell systems with improved properties and to establish methods to culture producer cell lines with high specific productivities as a basis for high yield processes.

Since most biopharmaceutical products are proteins that are secreted from the cells during the production process, the secretory transport machinery of the production cell line is another interesting target for novel host cell engineering strategies.

Protein secretion is a complex multi-step mechanism: Proteins destined to be transported to the extracellular space or the outer plasma membrane are first co-translationally imported into the endoplasmic reticulum. From there, they are packed in lipid vesicles and transported to the Golgi apparatus and finally from the trans-Golgi network (TGN) to the plasma membrane where they are released into the culture medium.

The yield of any biopharmaceutical production process depends largely on the amount of protein product that the producing cells secrete per time when grown under process conditions. Many complex biochemical intracellular processes are necessary to synthesize and secrete a therapeutic protein from a eukaryotic cell. All these steps such as transcription, RNA transport, translation, post-translational modification and protein transport are tightly regulated in the wild-type host cell line and will impact on the specific productivity of any producer cell line derived from this host. Many engineering approaches have employed the growing understanding of the molecular networks that drive processes such as transcription and translation to increase the yield of these steps in protein production. However, as for any multi-step production process, widening a bottle-neck during early steps of the process chain possibly creates bottle-necks further downstream, especially post translation. Up to a certain threshold, the specific productivity of a production cell has been reported to correlate linearly with the level of product gene transcription. Further enhancement of product expression at the mRNA level, however, may lead to an overload of the protein synthesis, folding or transport machinery, resulting in intracellular accumulation of the protein product. Indeed, this can be frequently observed in current manufacturing processes.

One recent approach to increase the secretion capacity of mammalian cells is the heterologous overexpression of the transcription factor X-box binding protein 1 (XBP-1). XBP-1 is one of the master-regulators in the differentiation of plasma cells, a specialized cell type optimized for high-level production and secretion of antibodies (Iwakoshi et al., 2003). XBP-1 regulates this process by binding to the so called ER stress responsive elements (ERSE) and unfolded protein response elements (UPRE) within the promoters of a wide spectrum of secretory pathway genes, resulting in (i) a physical expansion of the ER, (ii) increased mitochondrial mass and function, (iii) larger cell size and (iv) enhanced total protein synthesis (Shaffer et al., 2004).

Recently, attempts were described to increase protein secretion by overexpressing XBP-1 in non-plasma cells, especially production cell lines. In CHO-K1 cells, the production level of two reporter proteins (secreted alkaline phospatase (SEAP) and secreted alpha-amylase (SAMY) was shown to increase after XBP-1 introduction in CHO-K1 cells (Tigges and Fussenegger, 2006). A follow-up study than demonstrated the applicability of this approach for commercial manufacturing of recombinant proteins using CHO and NS0 cell lines and conditions relevant for industrial production (Ku et al., 2007). Furthermore, the patent application WO2004111194 by Ailor Eric describes the overexpression of XBP-1 or ATF6 for the generation of highly productive cell lines.

These studies prove that there is a post-translational bottle-neck in mammalian cell based production processes. With respect to industrial application, they open the exiting perspective to bypass this bottle-neck by genetic engineering through introducing a transgene that exerts its action post-translationally in the secretory pathway. This appears of particular relevance as the use of the latest generation of highly efficient expression vectors might lead to an overload of the protein-folding, -modification and transport machinery within the producer cell line, thus reducing its theoretical maximum productivity. The co-introduction of XBP-1 or another heterologous protein with secretion enhancing activity could overcome this limitation.

The present application describes a correlation between elevation of the specific productivity and XBP-1 expression (FIG. 1), meaning that cells with the highest level of XBP-1 display the highest antibody productivity. Consequently, the pre-requisite for successful engineering of host cells for commercial manufacturing of therapeutic proteins will be to obtain cells expressing XBP-1 at high levels.

However, counteracting the desirable effect of XBP-1 on the specific productivity, several lines of evidence reported within the present application demonstrate that XBP-1 confers a growth and survival disadvantage to the cells and thus the generation of stable IgG producing CHO cells expressing high amounts of heterologous XBP-1 proved to be difficult:

The present invention provides for the first time quantitative data showing that heterologous expression of XBP-1 indeed leads to reduced survival in colony formation assays (CFA).

In these assays, adherently growing CHO-K1 cells are transfected with either XBP-1 or empty control plasmids, the cells are seeded in dishes and subjected to selection pressure. Under these conditions, only those cells survive which have the expression constructs stably integrated into their genomes. These cells than grow up to form colonies which can than be counted and this number can be used to quantify the combined parameters cell survival and colony growth. In this assay, heterologous expression of XBP-1 in CHO-K1 cells leads to a significant decrease in the number of colonies compared to cells transfected with an empty expression construct (‘empty vector’ and ‘--/--’; FIGS. 4a and b). This result was reproducibly obtained with different XBP-1 expression constructs, including mono- and bi-cistronic expression plasmids. In all experiments, introduction of XBP-1 resulted in markedly less colonies compared to control experiments, thus confirming that introduction of XBP-1 induces apoptotic cell death.

In addition to the enhanced risk of apoptosis, another problem is that XBP-1 leads to reduced cell growth:

Following stable transfection of suspension cells, such as CHO-DG44, with XBP-1, we noted that only very few cell lines grew up after selection and sub-cloning and only a small fraction of these cell lines did express detectable amounts of XBP-1. This can be explained by a combination of two effects: The negative selection pressure hindering the survival of cells expressing XBP-1 at high levels and the reduced growth of XBP-1 positive cells. In heterogenous cell populations, this results in the overgrowth of slower growing XBP-1 positive cells by faster growing XBP-1 negative or low-expressing clones, leading to a continuous decline in the proportion of XBP-1 expressors in the cell pool.

Also the steps of limited dilution and single-cell cloning represent conditions where the cells are exposed to selective pressure and stress potentially inducing apoptosis and therefore might equally lead to a loss of XBP-1 high-expressors. This would be a crucial limitation to the applicability of this secretion engineering approach. Besides, timelines in industrial cell line development are strict and competitive, creating a demand for fast growing cells. Consequently, after the steps of selection or re-cloning, usually the first cells which grow up are picked for further expansion and there is no time to wait for slower growing cells, even if they had higher XBP-1 levels.

As a consequence, it is difficult to obtain stable XBP-1-transgenic cell clones.

Also on the clonal level, reduced growth properties represent a serious problem: In fed-batch processes, one of the most widely used culture formats for protein production, XBP-1 expressing clonal cell lines reach significantly lower maximal cell densities compared to control cells (FIG. 2). In a commercial manufacturing process, this means a reduction in the integral of viable cell concentration over time (IVC) and consequently lower final yields of the recombinant protein product.

Furthermore, this growth disadvantage conferred by XBP-1 could result in a negative selection pressure on the relevant clonal cell populations resulting in an increased likelihood of instable phenotypes in long-term serial cultivations. The currently most prominent regime for large scale manufacturing of proteins from mammalian cells starts from thaw of a working cell bank and includes establishing serial cultures in spinner flasks or shake flasks as a typical industrial inoculum setting. Several scale-ups can then be performed to expand cultures to the final bioreactor volume of usually more than 5000 L. This means several batches are generated from a single primary seed culture post working cell bank thaw. Therefore, to enable long campaigns in large scale manufacturing the minimal requirement for the maximum in vitro cell age post thaw of WCB can be more than 100 days. It is therefore crucial to ensure phenotypic and genotypic long-term stability, meaning that engineered producer cell lines, containing XBP-1 or one or several other transgenes, do not display changes in their phenotype with regard to transgene expression level, growth and specific productivity.

However, the negative pressure conferred by XBP-1 will favor the occurrence of genetic and phenotypic instability, as every cell which looses XBP-1 expression by either silencing or deletion of the XBP-1 expression cassette will gain a growth and survival advantage and will within few passages prevail within the culture.

Taken together, there is a clear need for improving the secretory capacity of host cells for recombinant protein production. With the current trend towards high-titer processes and more sophisticated expression enhancing technologies, post-translational bottle necks will become the evident rate-limiting steps in protein production and hence will draw increasing attention to secretion engineering approaches. However, one major challenge to these approaches is to prevent a concomitant growth-inhibitory and/or apoptotic response of the producer cell.

The present invention describes a novel and innovative method for increasing recombinant protein production.

The data of this application provide quantitative evidence that introduction of a secretion-enhancing transgene encoding a protein whose expression or activity is induced during the cellular processes of plasma cell differentiation, unfolded protein response (UPR) or endoplasmic reticulum overload response (EOR) in producer cell lines surprisingly results in a reduction in cell growth (FIG. 2) and enhanced apoptosis, as shown for the transcription factor XBP-1 (FIG. 4a).

In the present invention, we furthermore demonstrate that it is possible to circumvent this problem by co-expression of a second transgene with anti-apopototic function, such as the X-linked inhibitor of apoptosis (XIAP) or Bcl-XL.

In colony formation assays, XBP-1 expression led to a dramatic reduction in the number of colonies formed. However, by co-expression of an anti-apoptotic protein together with XBP-1, it was possible not only to restore but to increase colony numbers (as shown for XIAP, FIG. 4b). These data prove that inhibition of the apoptotic pathway in XBP-1 transfected cells is a suitable and effective means to overcome the survival disadvantage inherent to this secretion engineering approach.

In the present application we show data suggesting a direct correlation between XBP-1 expression level and enhancement of specific protein productivity in CHO-derived IgG producer cell lines (FIG. 1). For industrial applications, it would therefore be desirable to generate cell lines with high XBP-1 levels.

Whereas in a classical approach XBP-1 transfection of IgG producing CHO cells results in only few monoclonal cell lines with detectable XBP-1 levels, specific productivities, and titers in fed-batch cultures which are enhanced over the original IgG producing CHO cell line, the novel approach as described by the present invention results in monoclonal cell lines with higher XBP-1 levels, enhanced specific productivities, prolonged viabilities and higher titers in fed-batch processes (FIG. 3).

As a first major advantage, the present invention provides a strategy allowing for the generation of XBP-1 high-expressing cells by preventing growth reduction and apoptosis induced by XBP-1 over-expression.

We provide data showing that high XBP-1 expression leads to reduced cell growth and survival. However as we show in the present invention, the combination of secretion engineering by genes encoding proteins whose expression or activity is induced during one of the cellular processes of plasma-cell differentiation, unfolded protein response (UPR), or endoplasmic reticulum overload response (EOR), e.g. XBP-1, and anti-apoptitic cell engineering offers the possibility to compensate the increased sensitivity of such cells, e.g. XBP-1 expressing cells, by preventing them from entering into apoptosis. This approach allows even stable transfectants with high levels of secretion enhancing gene products such as XBP-1 to survive and thus enables the generation of high-expressing cell lines.

The second major advantage—which is linked with the first—is the generation of cells with markedly increased secretory capacity.

The present invention demonstrates a direct correlation between the level of XBP-1 expression and the cellular production capacity (FIG. 1). Thus, by enabling the survival of cells with high XBP-1 levels the method described in the present invention provides a means to generate cells with enhanced specific productivity.

In the present application, we furthermore provide data showing that the specific productivity of stable IgG secreting cell pools containing both XBP-1 and an anti-apoptotic gene is enhanced. In line with previous publications on XBP-1 (Tigges and Fussenegger, 2006), the specific productivity of IgG producing CHO cells is moderately elevated upon expression of XBP-1 alone, however the effect is much more pronounced in cells containing XBP-1 together with XIAP (FIG. 5) or Bcl-XL (FIG. 6). Notably, expression of an anti-apoptosis gene alone does not lead to a significant alteration in antibody productivity in stable cell pools (FIG. 5a), whereas concomitant expression of XBP-1 and XIAP leads to pools expressing markedly higher amounts of an antibody product.

These data indicate that the combination of both transgenes represents a clear advantage over the single-gene engineering approach and allows to explore the full potential of XBP-1 mediated secretion enhancement.

The third major advantage of the present invention is the increase of overall product yield in production processes by integration of secretion enhancement and increase in IVC:

In biopharmaceutical production processes, the overall yield is determined by two factors: the specific productivity (P_{spec}), of the host cell and the IVC, the integral of viable cells over time which produce the desired protein. This correlation is expressed by the following formula: Y=P_{spec}*IVC. Standard approaches to improve product yield have therefore aimed to increase either the production capacity of the host cell or viable cell densities in the bioreactor. The method of the present invention describes a combinatorial approach addressing both of these parameters at the same time by co-introduction of both, specific secretion enhancing genes which however confer a growth and/or survival disadvantage to the cell as well as anti-apoptotic genes.

Another advantage of the present invention is the improvement of long-term stability of XBP-1 expressing cell lines:

Co-introduction of an anti-apoptotic gene such as XIAP or Bcl-XL compensates the growth disadvantage in XBP-1 expressing cells. Thereby, it reduces the negative selective pressure on XBP-1 positive cell lines and thereby lowers the risk of genetic and/or phenotypic instability.

A further major advantage of the present invention is the transferability to anti-apoptotic genes in general.

In the present invention, we provide data indicating that the unexpected negative effect of XBP-1 on cell growth and survival can be counteracted by co-expression of both, XIAP and Bcl-XL (FIGS. 5 and 6).

It is important to note that XIAP and Bcl-XL are members of two protein families with different mechanisms of action which can even be part of different apoptotic pathways:

XIAP is the best studied member of the IAP (inhibitors of apoptosis) family of proteins, known and potent inhibitors of caspases which are involved in both, the mitochondrial and the so-called extrinsic apoptotic pathways (Reed, 2000). IAP proteins are characterized by one or more copies of an about 70-amino acid motiv, termed BIR (baculovirus IAP repeat) domains. Via these domains, IAP proteins are able to bind to and inhibit the enzymatic activity of caspase-3, -7 and -9, known effectors of the apoptotic response. In humans, six members of the IAP family have been identified so far, including XIAP, cellular inhibitor of apoptosis 1 and 2 (cIAP1, cIAP2), neuronal inhibitor protein (NIAP), living and surviving.

In contrast, Bcl-XL belongs to the Bcl-2 family of proteins which are implicated in the mitochondrial pathway of apoptosis. This family comprises over 20 members with pro- and anti-apoptotic functions. The proteins with anti-apoptotic activity include Bcl-2, Bcl-XL, Mcl-1, Bfl-1, Bcl-W and Diva/Boo. Based upon the structural features, it has been suggested that Bcl-2 proteins might act by inserting into the outer mitochondrial membrane where they regulate membrane homeostasis and prevent uncontrolled release of cytochrome c, a central player in the intrinsic apoptotic pathway (Hengartner, 2000).

In the present invention we show that members of both families of anti-apoptotic proteins, such as Bcl-XL and XIAP, can equally be used in combination with XBP-1 to prevent apoptosis and/or growth reduction and thereby synergistically enhance recombinant protein production. These results suggest, that the basic principle is to prevent apoptosis induced upon XBP-1 over-expression can be exerted not only by Bcl-XL and XIAP, but by all anti-apoptotic members of the two protein families, if not by all proteins with anti-apoptotic function.

Notably, there seem to be combinations of XBP-1 and anti-apoptosis genes that are more effective than others. Comparing XIAP and the Bcl-XL mutant, XIAP had the strongest effect on cell survival in colony formation assays and cell pools expressing XBP-1 and XIAP showed an over 50% increase in specific antibody productivities compared to cells expressing XIAP alone (FIG. 5a). Co-transfection of the Bcl-XL mutant and XBP-1, however, still resulted in a significant increase in antibody productivity, but this was less pronounced and only about 20% higher than in cells expressing only Bcl-XL (FIG. 6). In addition, we also performed the same set of experiments using wild type Bcl-XL, however this transgene was less effective than the Bcl-XL mutant. This might in part be attributed to the expression level, as it has been published that high amounts of Bcl-XL within the cell are required to efficiently protect the cells against apoptosis. Therefore, amplification of the Bcl-XL gene or the use of Bcl-XL mutants with prolonged protein stability might be required to achieve the full protective effect.

But even the Bcl-XL mutant proved to be less efficient than XIAP in protecting XBP-1 expressing cells from apoptotic cell death, indicating that it is important to find out the most effective combination of secretion-enhancing and anti-apoptosis genes. The most effective combination identified in the present application is a combination of XBP-1 and XIAP.

It is an essential aspect of the present invention that the secretion enhancing genes described in the present invention convey a reduction in cell growth and/or a survival disadvantage. The secretion enhancing genes of the present invention like XBP-1 are linked as a group by the common physiological context in which they exert their function, namely secretory cell differentiation and the unfolded protein response (UPR)/endoplasmic reticulum overload response (EOR) responses, and which as a common final outcome lead to growth arrest and apoptosis.

As mentioned above, XBP-1 was described to play a crucial role in regulating the transition from B-cells to terminally differentiated and secretion-competent plasma cells. In addition, it was recently demonstrated in tissue-specific rescue experiments using XBP-1 knockout mice that XBP-1 is also necessary for full biogenesis of the secretory machinery of pancreatic and salivary gland acinar cells (Lee et al., 2005).

The process of terminal differentiation, such as the maturation from lymphocyte to plasma cell, is usually regarded an apoptosis-like program, during which the cell loses its proliferative capacity to give rise to a terminally differentiated secretory cell. In fact, nearly all cell types specifically designed for high-level protein secretion (e.g. glandular cells, pancreatic beta cells) are terminally differentiated, are not able to proliferate and have a limited life-span before ultimately undergoing programmed cell death (Chen-Kiang, 2003). Notably, XBP-1 does not only regulate secretory cell differentiation but also plays an important role in the unfolded protein response (UPR) (Brewer and Hendershot, 2005). The UPR represents a complex signal transduction network activated by accumulation of unfolded or incorrectly processed proteins in the endoplasmic reticulum (ER). The UPR coordinates adaptive responses to this stress situation, including induction of ER resident molecular chaperone and protein foldase expression to increase the protein folding capacity of the ER, induction of phospholipid synthesis, attenuation of general translation, and upregulation of ER-associated degradation to decrease the unfolded protein load of the ER. Upon severe or prolonged ER stress, the UPR ultimately induces apoptotic cell death (Schroder, 2006).

Therefore, further secretion enhancing genes of the present invention include, besides XBP-1, all direct inducers of XBP-1 during the processes of plasma cell differentiation, UPR and the ER overload response (EOR). This includes all proteins which positively regulate XBP-1 either by binding to its promoter thereby inducing transcription of the XBP-1 gene (e.g. IRF4) or by regulating its activity post-transcriptionally, e.g. by inducing splicing of the XBP-1 mRNA into its active form, as described for the transmembrane nuclease IRE.

As a transcription factor, XBP-1 exerts its function by binding to distinct sequence elements, called ER-stress response elements (ERSE), in the promoter regions of target genes thereby regulating their expression. Two ERSE motives and a UPRE (“unfolded protein response element”) have been described that are found in the promoters of several hundred genes, including phosphodisulfide isomerase (PDI) and the chaperone binding protein (BiP). Interestingly, both proteins have been used for cell engineering in the past, with various success.

It is thus a major embodiment of the present invention that concomitant expression of these genes or other XBP-1 targets together with anti-apoptotic genes represents a superior strategy to overcome the limitations of the single-gene approaches.

Furthermore, it is a preferred embodiment of present invention that the method described in the present application extends to other transcription factors involved in UPR and/or EOR, such as ATF6 and CHOP, and possibly even to all proteins implicated in these two processes, including eIF2-alpha, PERK and PKR.

The invention describes a method to generate improved eukaryotic host cells for the production of heterologous proteins by combining secretion-enhancing and anti-apoptotic cell engineering, whereby the secretion enhancing gene is a gene encoding a protein whose expression or activity is induced during one of the following cellular processes: plasma-cell differentiation, unfolded protein response (UPR), endoplasmic reticulum overload response (EOR).

This novel approach leads to increased overall protein yields in production processes based on eukaryotic cells by influencing both, the specific productivity and the integral of viable cells over time, by improving the secretory capacity of the cells and simultaneously reducing apoptosis during fermentation.

The approach described here will thereby reduce the cost of goods of such processes and at the same time reduce the number of batches that need to be produced to generate the material required for research studies, diagnostics, clinical studies or market supply of a therapeutic protein. The invention will furthermore speed up drug development as often the generation of sufficient amounts of material for pre-clinical studies is a critical work package with regard to the timeline.

The invention can be used to increase the protein production capacity of all eukaryotic cells used for the generation of one or several specific proteins for either diagnostic purposes, research purposes (target identification, lead identification, lead optimization) or manufacturing of therapeutic proteins either on the market or in clinical development.

As secreted and transmembrane proteins share the same secretory pathways and are equally imported into the ER, processed and transported in lipid-vesicles as secreted proteins, the present invention might not only be applicable to enhance protein secretion, but also to increase the abundance of transmembrane proteins on the cell surface. Therefore, the method described herein can also be used for academic and industrial research purposes which aim to characterize the function of cell-surface receptors. E.g. it can be used for the production and subsequent purification, crystallization and/or analysis of surface proteins. Furthermore, transmembrane proteins generated by the described method or cells expressing these proteins can be used for screening assays, e.g. screening for substances, identification of ligands for orphan receptors or search for improved effectiveness during lead optimization. This is of crucial importance for the development of new human drug therapies as cell-surface receptors are a predominant class of drug targets.

Moreover, it might be advantageous for the study of intracellular signalling complexes associated with cell-surface receptors or the analysis of cell-cell-communication which is mediated in part by the interaction of soluble growth factors with their corresponding receptors on the same or another cell.

### SUMMARY OF THE INVENTION

In summary, the present invention provides a method for enhancing protein production from eurkaryotic, especially mammalian cells by co-introduction of secretion-enhancing and anti-apoptotic transgenes into the same cell, whereby the secretion enhancing gene confers a growth and/or survival disadvantage to said cell.

This approach allows not only to combine the known advantages of both single-gene engineering approaches, but in addition it represents the solution to the as yet unresolved problem of growth reduction and/or increased apoptosis triggered by over-expression of genes involved in a cellular stress response, such as XBP-1, in the unfolded protein response, its transcriptional target genes or its direct upstream regulators.

In the present invention, we surprisingly demonstrate for the first time that over-expression of XBP-1 leads to a reduction in cell growth and survival in cell lines relevant for therapeutic protein production. This effect of reduction in cell growth and survival is surprising, because so far, a direct apoptosis induction by XBP-1(s) overexpression has never been reported in the prior art. To date, only the UPR mediators activating transcription factor 6 (ATF6) and Inositol-requiring enzyme 1 (IRE1) were shown to be directly involved in apoptosis induction: ATF6 induces apoptosis via transcriptional activation of pro-apoptotic protein CHOP (also known as growth arrest and DNA-damage-inducible protein GADD153) (Zinszner et al., 1998; Yoshida et al., 1998) and IRE1 via TNF receptor associated factor 2 (TRAF2) mediated activation of the c-Jun amino-terminal kinase (JNK) pathway (Urano et al., 2000). The branching point with link to the apoptotic signalling cascade was thereby shown to be at IRE1α which is upstream of the XBP-1 in the signalling cascade. These data prove that the demonstrated surprising apoptosis induction upon XBP-1(s) overexpression can not be transmitted by IRE1α.

Furthermore, the effect of reduction in cell growth and survival upon XBP-1 over-expression is surprising, because none of the studies known in the prior art using XBP-1(s) to enhance the productivity of producer cell lines reported on negative impacts of XBP-1(s) overexpression (Campos-da-Paz et al., 2008; Ku et al., 2007; Ohya et al., 2007; Tigges and Fussenegger, 2006).

This disadvantage in cell growth and survival upon XBP-1 over-expression can be more than compensated by co-introduction of genes with anti-apoptotic function, such as XIAP or Bcl-XL, which play part in the “external” as well as the “intrinsic” mitochondrial pathways.

Furthermore, in the present invention we provide data showing that co-expression of transgenes with anti-apoptotic function enables survival of cells expressing high amounts of XBP-1, thereby leading to populations with significantly higher specific productivities of heterologous proteins compared to all populations that have been generated without introducing the anti-apoptotic gene. In addition, this also allows for the generation of clonal cell lines with markedly increased specific productivities due to high-level XBP-1 expression.

Moreover, the combination of XBP-1 and anti-apoptosis genes like XIAP or Bcl-Xl provides a strategy for synergistic enhancement of overall protein yields by integrating both, improvement of productivity and prolonged cell survival resulting in higher IVCs during the production process.

Taken together, the data shown in the present invention demonstrate the applicability of both, XIAP and Bcl-XL/BCL-XL mutant to enhance the specific productivity of antibody producer cells in combination with XBP-1/secretion enhancing genes conferring reduced growth and/or survival. Both proteins, XIAP and Bcl-X/BCL-XL mutant, are known antagonists of apoptosis, but XIAP acts by inhibiting caspases whereas Bcl-X/BCL-XL mutant exerts its apoptotic role by preventing the uncontrolled efflux of apoptogenic molecules from mitochondria. Despite these different modes of action, both proteins are effective in this multigene-engineering approach of the present invention, thereby demonstrating the broad applicability of this approach for any protein with anti-apoptotic function.

Notably, the extend of enhancement regarding increase of specific antibody productivities achieved by using XIAP is stronger as with Bcl-XL and Bcl-XL mutant.

The specific antibody productivities of the wildtype form of Bcl-XL together with XBP-1 has lower increase in the specific antibody productivities than with the Bcl-XL deletion mutant, which is most likely to be due to higher protein levels of the mutant within the cell as a result of improved protein stability.

The present invention is not obvious from the prior art.

Until now, multigene metabolic engineering approaches have been mainly directed to control of cell cycle progression, as one of the key-regulatory mechanisms within a cell. For example, a tri-cistronic expression cassette comprising the reporter protein SEAP together with the cell-cycle regulator p21 and the differentiation factor C/EBP-alpha (CAAT-enhancer binding protein alpha) was shown to lead to sustained growth arrest and higher specific productivities (Timchenko et al., 1996).

A second example for “multigene metabolic engineering” technology was the use of a p27-Bcl-XL encoding bi-cistronic expression unit, which resulted in higher expression levels in CHO cells compared to control cells (Fussenegger et al., 1998).

Another approach was to combine two genes involved in the same cellular process, as demonstrated for the co-expression of the two anti-apoptotic genes Aven and Bcl-XL (Figueroa, Jr. et al., 2004), in order to gain more effective control over the mechanism of regulated cell death.

The present invention represents the first example for a combinatorial approach, integrating the advantages of targeting secretion enhancing genes and the apoptosis pathway within the same cell, whereby the secretion enhancing gene is a gene encoding a protein whose expression or activity is induced during one of the following cellular processes: plasma-cell differentiation, unfolded protein response (UPR), endoplasmic reticulum overload response (EOR).

The surprising and unexpected working model of the present invention identifies the combined introduction of secretion-enhancing and anti-apoptosis genes as a strategy to enhance therapeutic protein production by two mechanisms: (i) by facilitating/enabling the survival of XBP-1 high-expressors thus allowing to make use of the full potency of this approach to enhance the cell's specific productivity and (ii) by encompassing the advantages of increasing cell viability in protein production processes.

### DESCRIPTION OF THE FIGURES

FIG. 1: Korrelation XBP-1 Expression and Productivity

(a) Western blot of nuclear extracts from the same clones to confirm XBP-1 expression. Lysates from transiently transfected cells served as negative (Mock) and positive control (48h XBP1).

(b) The specific productivities of antibody producing CHO-DG44 cells (parental), one mock clone (E5) and two monoclonal XBP-1 expressing cell lines E_{—}23 and E_{—}27 was calculated during serial cultivation over five (mock) or 11 passages. The values are represented as mean values relative to the specific productivity of the parental cell line, error bars represent the standard deviations of the serial passages.

FIG. 2: Reduction in Maximal Cell Densities

A fed-batch production run was performed in shake flasks (n=3). Viable cell count was assessed by the CEDEX system (Innovatis AG, Bielefeld, Germany).

FIG. 3: Flow Chart Schematic Comparing Classic Versus Novel XBP-1-Based Cell Engineering Approach

This scheme summarizes the advantages of the novel approach as described in the present invention in comparison to the classic XBP-1-based cell engineering approach.

FIG. 4: Colony Forming Assay (CFA) with Monocistronic and Bicistronic Expression Constructs (Empty Vector=100%)

Adherent growing CHO-K1 cells were transfected with an empty vector and a monocistronic vector expressing the active form of XBP-1(s). After 24 h the cells trypsinated and 1×10⁵ cells were transferred to 9 cm Petri-dishes and allowed to adhere for 24 h under culture conditions. The selection antibiotic puromycin was added and the dishes incubated for 12 days. After staining the colonies were counted manually. All experiments were done in duplicates.

(a) The colony count in percent of the control vector is shown for the monocistronic expression vectors (control black bar, XBP1 grey bar).

(b) For bicistronic vectors the colony count in percent of control is shown. The assay was performed as for the monocistronic vector constructs. Here, CHO-K1 cells were transfected with either empty vector (--/--, black bar), a vector coding for XBP-1(s) in the second cistron (--/XBP1, grey bar) or with the gene combination comprising the anti-apoptotic gene in the first and the secretion enhancer in the second cistron (XIAP/XBP1, cross structured bar).

FIG. 5: Specific Productivity of Transfected MAB Producing Cells with XIAP

A therapeutic IgG antibody producing CHO-DG44 clone was transfected with either empty IRES containing vector (--/--, black bar), a vector coding for XBP-1(s) in the second cistron (--/XBP1, grey bar), a vector coding for the anti-apoptotic gene XIAP in the first cistron (XIAP/--, vertically structured bar) or with the gene combination comprising the anti-apoptotic gene in the first and the secretion enhancer in the second cistron (XIAP/XBP1, cross structured bar).

(a) The specific productivity of three pool populations was determined over three consecutive passages and is shown as mean values.

(b) After a subcloning procedure the IgG concentration per well was analyzed. To compare the data colony size was divided in large and medium sized colonies by microscopic inspection. The median IgG concentration for each genotype is shown. The bars represent a dataset with at least 19 clones per genotype.

FIG. 6: Specific Productivity MAB Producing Cells Transfected With Further Anti-Apoptotic Gene with BCL-XL Mutant

The same antibody producing CHO-DG44 clone as in FIG. 5a) was transfected with bicistronic plasmids coding only for mutant of BclxL in the first cistron (BclxLₘᵤₜ/--, black bar) or again combined with XBP1 (BclxLₘᵤₜ/XBP1, grey bar).

The specific productivity of three pool populations was determined over three consecutive passages and is shown as mean value.

FIG. 7: Elevated Apoptosis Induced by XBP-1 and Rescue by Concomitant XIAP Expression

CHO-K1 cells were transfected either with the empty plasmid (Mock), XBP-1(s), XIAP or both plasmids together (XBP-1/XIAP). The data show the relative apoptosis rate compared to mock-transfected cells 48 h after transfection as determined by annexin-V/PI staining. The data represent the mean of three independent experiments run in triplicate samples. The apoptotic rate in mock cells was set 100%.

FIG. 8: Decreasing XBP-1 Expression and Specific Productivities in Long Term Cultures

The two stable XBP-1(s) expressing cell lines E23 (black) and E27 (grey) are cultivated for 35 passages.

(A) XBP-1 mRNA levels are measured in an early (P10) and in a later passage (P35). Beta tubulin was used for normalization.

(B) Specific productivities determined from supernatant samples of the same cultures at passages 10 and 35.

FIG. 9: Increased Expression of XBP-1 in Engineered Cells

XBP-1 mRNA transcript levels in cell populations stably transfected with empty vector (Mock, black bar) or expression constructs encoding either XBP-1 alone (grey) or XBP-1 and XIAP (XBP-1/XIAP; striated bar). The bars represent mean values of three cell populations and are depicted relative to the level measured in Mock cells. All PCR measurements are done in triplicates using beta-tubulin for standardization.

### DETAILED DESCRIPTION OF THE INVENTION

The general embodiments “comprising” or “comprised” encompass the more specific embodiment “consisting of”. Furthermore, singular and plural forms are not used in a limiting way.

Terms used in the course of this present invention have the following meaning

The term “secretion-enhancing gene” refers to all proteins which lead to an increase in the amount of protein in the culture medium when overexpressed in protein secreting cells. This function can e.g. be quantitatively measured by ELISA detecting the protein-of-interest in the cell culture fluid from cells which have been transfected with the secretion-enhancing gene compared to untransfected cells.

More specifically, the term “secretion-enhancing gene” includes all genes and proteins which are induced or activated during the unfolded protein response (UPR) and the ER overload response (EOR) as well as plasma cell differentiation. Even more specifically, this term comprises all genes which contain ER-stress response elements (ERSE-1 or -2) as represented in SEQ ID NO 9 or 10 or one or more unfolded protein response elements (UPRE) as represented in SEQ ID NO 11 and 12 within their respective promoters.

The term “growth and/or survival disadvantage” means the effect of a transgene on the growth properties of cells which is measurable in a colony formation assay and/or the performance of a cell containing a transgene during fed-batch cultivation:

### Colony Formation Assay (CFA)

Adherent CHO-K1 cells are transfected with an expression construct encoding a transgene and a puromycin resistance gene or an empty vector as control. 24 h after transfection, the cells are trypsinated and 1×10⁵ cells are transferred to a 9 cm Petri dish containing finally 12 ml fresh culture medium. The cells are allowed to adhere for 24 h under culture conditions before adding the selection antibiotic puromycin at a final concentration of 5-15 mg/L. The dishes are cultured at 37° C. and 5% CO2 atmosphere for 12 days. Next, the colonies are fixed with ice cold Aceton/Methanol (1:1) for five minutes, then stained with Giemsa (1:20 in dest. Water) for 15 minutes and the colonies are counted manually for analysis. A growth and/or survival disadvantage would be detected as a reduced number of colonies formed and/or reduced sizes of the colonies.

### Fed Batch Cultivation:

Cells containing the transgene to be analysed and untransfected control cells are subjected to a fed-batch process. For this purpose, cells are seeded at 3×10⁵ cells/ml into 1000 ml shake flasks in 250 ml of production medium. The cultures are agitated at 120 rpm in 37° C. and 5% CO₂ which is later reduced to 2% as cell numbers increase. Culture parameters including pH, glucose and lactate concentrations are determined daily and pH is adjusted to pH 7.0 using NaCO₃ as needed and feed solution is added every 24 hrs. Cell densities and viability are determined by trypan-blue exclusion using an automated CEDEX cell quantification system (Innovatis AG, Bielefeld, Germany). A transgene conferring a growth and/or survival disadvantage would lead to reduced maximal cell densities of the cells carrying said transgene and/or decreased IVC's over the production process.

The term “ERSE” stands for “ER-stress responsive element”. The ERSEs 1 and 2 (SEQ ID NO 9 and 10) are DNA sequence motives in promoter regions of genes which serve as specific binding sites for transcription factors.

The term “UPRE” stands for “unfolded protein response element” and refers to a 8 bp DNA sequence motive contained in the promoter regions of genes which serves as specific binding sites for transcription factors (SEQ ID NO 11 and 12).

The term “secretion engineering” describes the method of introducing a secretion-enhancing gene into a cell with the purpose of increasing protein secretion. This includes the introduction of a secretion-enhancing gene into a production host cell as well as the improvement of cells already expressing a heterologous protein-of-interest.

The term “XBP-1” equally refers to the XBP-1 DNA sequence and all proteins expressed from this gene, including XBP-1 splice variants. Preferentially, XBP-1 refers to the human XBP-1 sequence and preferrably to the spliced and active form of XBP-1, also called “XBP-1(s)” (SEQ ID NO 1 and 2).

The term “anti-apoptotic gene” or “anti-apoptosis gene” includes all genes and proteins which lead to an inhibition or delay in apoptotic cell death when over-expressed in cells. Functionally, heterologous expression of “anti-apoptosis” genes in cells results in inhibition and/or delay of caspase activation, especially the proteolytic activation of the effector caspases 3 and 9, and consequently inhibition and/or delay of apoptotic cell responses such as DNA laddering and AnnexinV exposure.

More specifically, the term includes all members of the IAP and Bcl-2 protein families, namely XIAP, cellular inhibitor of apoptosis 1 and 2 (cIAP1, cIAP2), neuronal inhibitor protein (NIAP), living and surviving for the IAP family as well as over 20 proteins which contain one or more Bcl-2 homology (BH) domains, including without limitation Bcl-2, Bcl-XL, Mcl-1, Bfl-1, Bcl-W and Diva/Boo.

The term “BIR” domain means a conserved protein domain of about 70 amino acids. BIR stands for ‘Baculovirus Inhibitor of apoptosis protein repeat’. It is found repeated in inhibitor of apoptosis proteins (IAPs), and in fact it is also known as IAP repeat. These domains characteristically have a number of invariant residues, including three conserved cysteines and one conserved histidine that coordinate a zinc ion. They are usually made up of 4-5 alpha helices and a three-stranded beta-sheet. The BIR domain has the pfam number pfam00653, whereby pfam numbers define unique entries in the “Conserved Domains” database at NCBI. The BIR consensus sequence is represented as SEQ ID NO 13.

The members of the “Bcl-2 family” share one or more of the four characteristic domains of homology entitled the “Bcl-2 homology (BH) domains” (named BH1, BH2, BH3 and BH4). The BH domains have the pfam number pfam00452, whereby pfam numbers define unique entries in the “Conserved Domains” database at NCBI. The BH domains are known to be crucial for function, as deletion of these domains via molecular cloning affects survival/apoptosis rates. Most proteins in the Bcl-2 superfamily also harbour C-terminal signal-anchor sequences that target them predominantly to the outer mitochondrial membrane, endoplasmic reticular membrane and the outer nuclear envelope.

Examples of anti-apoptotic Bcl-2 family members characterized by comprising all four BH domains within their sequence include Bcl-2, Bcl-XL, Mcl-1, CED-9, A1 and Bfl-1. The Bcl-2 domain consensus sequence is represented as SEQ ID NO 14.

The term “XIAP” equally refers to the XIAP DNA sequence and all proteins expressed from this gene, including XIAP splice variants and XIAP mutants. XIAP mutants include without limitation mutants containing point mutations as well as insertion or deletion mutants, especially mutants generated by deletions of one or more BIR domains or by deletion of the C-terminal RING-domain. Preferentially, XIAP refers to the human XIAP sequence (SEQ ID NO 3 and 4).

The term “BCL-XL” denominates an inhibitor of the mitochondrial apoptotic pathway. It is known from the bcl-xL gene, that two different RNA molecules are produced, one of which codes for BCL-xL (long form) and one of which codes for BCL-xS (short form). The BCL-xS lacks a section of 63 amino acids found in the BCL-xL. BCL-xS has been shown to favor apoptosis, and therefore it is preferable to use a cDNA for expression of the BCL-xL rather than a genomic fragment.

A preferred sequence of BCL-xL protein is represented by SEQ ID NO 6, which is encoded by bcl-xL gene with the SEQ ID NO 5.

The term “BCL-xL mutant” denominates a protein derived from BCL-xL with improved anti-apoptosis properties, e.g. generated by deleting a non-conserved region between the BH3 and BH4 conserved regions and thus increasing the protein stability of the mutant protein variants (Chang et al., 1997; Figueroa et al., 2001). A preferred sequence of BCL-xL mutant protein is represented by SEQ ID NO 8, which is encoded by bcl-xL gene with the SEQ ID NO 7.

The term “derivative” in general includes sequences suitable for realizing the intended use of the present invention.

The term “derivative” as used in the present invention means a polypeptide molecule or a nucleic acid molecule which is at least 70% identical in sequence with the original sequence or its complementary sequence. Preferably, the polypeptide molecule or nucleic acid molecule is at least 80% identical in sequence with the original sequence or its complementary sequence. More preferably, the polypeptide molecule or nucleic acid molecule is at least 90% identical in sequence with the original sequence or its complementary sequence. Most preferred is a polypeptide molecule or a nucleic acid molecule which is at least 95% identical in sequence with the original sequence or its complementary sequence and displays the same or a similar effect on secretion as the original sequence.

Sequence differences may be based on differences in homologous sequences from different organisms. They might also be based on targeted modification of sequences by substitution, insertion or deletion of one or more nucleotides or amino acids, preferably 1, 2, 3, 4, 5, 7, 8, 9 or 10 amino acids. Deletion, insertion or substitution mutants may be generated using site specific mutagenesis and/or PCR-based mutagenesis techniques. The sequence identity of a reference sequence can be determined by using for example standard “alignment” algorithms, e.g. “BLAST”. Sequences are aligned when they fit together in their sequence and are identifiable with the help of standard “alignment” algorithms.

Furthermore, in the present invention the term “derivative” means a nucleic acid molecule (single or double strand) which hybridizes to other nucleic acid sequences. Preferably the hybridization is performed under stringent hybridization- and washing conditions (e.g. hybridisation at 65° C. in a buffer containing 5×SSC; washing at 42° C. using 0.2×SSC/0.1% SDS).

The term “derivatives” further means protein deletion mutants, phosphorylation or glycosylation mutants.

The term “activity” describes and quantifies the biological functions of the protein within the cell or in in vitro assays.

An example of how to measure “activity” of anti-apoptotic genes is to measure the proteolytic activation of the effector caspases-3 or -9, e.g. by detection of specific cleavage products in Western Blot experiments.

Another method to measure “activity” of anti-apoptotic genes is to measure the cellular processes which are characteristic for apoptosis such as DNA laddering which can be visualized in agarose gelelectrophoresis or AnnexinV-exposure on the cell surface.

“Activity” of a secretion-enhancing gene can be measured by transfecting the gene into a cell expressing a secreted protein-of-interest and measuring the amount of said protein in the cell culture fluid by ELISA. Cells that have been transfected with a secretion-enhancing gene will secrete more, preferably at least 20% more protein-of-interest compared to untransfected cells.

One method to measure the “activity” of XBP-1 is to perform band-shift experiments to detect binding of the XBP-1 transcription factor to its DNA binding site. Another method is to detect translocation of the active XBP-1 splice variant from the cytosol to the nucleus. Alternatively, XBP-1 “activity” can be indirectly confirmed by measuring induced expression of a bona fide XBP-1 target gene such as binding protein (BiP) upon heterologous expression of XBP-1. Another method to measure XBP-1 activity is to perform a luciferase assay using a DNA construct encoding the luciferase reporter gene controlled by a promoter containing XBP-1 binding sites. Increased activity in this assay would mean a 2-fold increase in the luciferase signal compared to an untransfected or mock-transfected control cell.

“Host cells” in the meaning of the present invention are cells such as hamster cells, preferably BHK21, BHK TK⁻, CHO, CHO-K1, CHO-DUKX, CHO-DUKX B1, and CHO-DG44 cells or the derivatives/progenies of any of such cell line. Particularly preferred are CHO-DG44, CHO-DUKX, CHO-K1 and BHK21, and even more preferred CHO-DG44 and CHO-DUKX cells. In a further embodiment of the present invention host cells also mean murine myeloma cells, preferably NS0 and Sp2/0 cells or the derivatives/progenies of any of such cell line. Examples of murine and hamster cells which can be used in the meaning of this invention are also summarized in Table 1. However, derivatives/progenies of those cells, other mammalian cells, including but not limited to human, mice, rat, monkey, and rodent cell lines, or eukaryotic cells, including but not limited to yeast, insect and plant cells, can also be used in the meaning of this invention, particularly for the production of biopharmaceutical proteins.

TABLE 1
Eukaryotic production cell lines

| CELL LINE | ORDER NUMBER |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX (=CHO duk⁻, CHO/dhfr⁻) | ATCC CRL-9096 |
| CHO-DUKX B11 | ATCC CRL-9010 |
| CHO-DG44 | (Urlaub et al., 1983) |
| CHO Pro-5 | ATCC CRL-1781 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |

Host cells are most preferred, when being established, adapted, and completely cultivated under serum free conditions, and optionally in media which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invitrogen), serum-free CHO Medium (Sigma), and protein-free CHO Medium (Sigma) are exemplary appropriate nutrient solutions. Any of the media may be supplemented as necessary with a variety of compounds examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that would be known to those skilled in the art. In the present invention the use of serum-free medium is preferred, but media supplemented with a suitable amount of serum can also be used for the cultivation of host cells. For the growth and selection of genetically modified cells expressing the selectable gene a suitable selection agent is added to the culture medium.

The term “protein” is used interchangeably with amino acid residue sequences or polypeptide and refers to polymers of amino acids of any length. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with like properties.

The term “polypeptide” means a sequence with more than 10 amino acids and the term “peptide” means sequences up to 10 amino acids length.

The present invention is suitable to generate host cells for the production of biopharmaceutical polypeptides/proteins. The invention is particularly suitable for the high-yield expression of a large number of different genes of interest by cells showing an enhanced cell productivity.

The term “gene” can equally refer to the gene, meaning the DNA sequence, as well as the protein product into which the DNA sequence is translated. The terms “gene” and “protein” can thus be used interchangeably. In the present invention, these terms refer preferrably to human genes and proteins, but included are equally homologous sequences from other mammalian species, preferably mouse, hamster and rat, as well as homologous sequences from additional eucaryotic species including chicken, duck, moss, worm, fly and yeast.

“Gene of interest” (GOI), “selected sequence”, or “product gene” have the same meaning herein and refer to a polynucleotide sequence of any length that encodes a product of interest or “protein of interest”, also mentioned by the term “desired product”. The selected sequence can be full length or a truncated gene, a fusion or tagged gene, and can be a cDNA, a genomic DNA, or a DNA fragment, preferably, a cDNA. It can be the native sequence, i.e. naturally occurring form(s), or can be mutated or otherwise modified as desired. These modifications include codon optimizations to optimize codon usage in the selected host cell, humanization or tagging. The selected sequence can encode a secreted, cytoplasmic, nuclear, membrane bound or cell surface polypeptide.

The “protein of interest” includes proteins, polypeptides, fragments thereof, peptides, all of which can be expressed in the selected host cell. Desired proteins can be for example antibodies, enzymes, cytokines, lymphokines, adhesion molecules, receptors and derivatives or fragments thereof, and any other polypeptides that can serve as agonists or antagonists and/or have therapeutic or diagnostic use. Examples for a desired protein/polypeptide are also given below.

In the case of more complex molecules such as monoclonal antibodies the GOI encodes one or both of the two antibody chains.

The “product of interest” may also be an antisense RNA.

“Proteins of interest” or “desired proteins” are those mentioned above. Especially, desired proteins/polypeptides or proteins of interest are for example, but not limited to insulin, insulin-like growth factor, hGH, tPA, cytokines, such as interleukines (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosisfactor (TNF), such as TNF alpha and TNF beta, TNF gamma, TRAIL; G-CSF, GM-CSF, M-CSF, MCP-1 and VEGF. Also included is the production of erythropoietin or any other hormone growth factors. The method according to the invention can also be advantageously used for production of antibodies or fragments thereof. Such fragments include e.g. Fab fragments (Fragment antigen-binding=Fab). Fab fragments consist of the variable regions of both chains which are held together by the adjacent constant region. These may be formed by protease digestion, e.g. with papain, from conventional antibodies, but similar Fab fragments may also be produced in the mean time by genetic engineering. Further antibody fragments include F(ab')2 fragments, which may be prepared by proteolytic cleaving with pepsin.

The protein of interest is preferably recovered from the culture medium as a secreted polypeptide, or it can be recovered from host cell lysates if expressed without a secretory signal. It is necessary to purify the protein of interest from other recombinant proteins and host cell proteins in a way that substantially homogenous preparations of the protein of interest are obtained. As a first step, cells and/or particulate cell debris are removed from the culture medium or lysate. The product of interest thereafter is purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin such as DEAE. In general, methods teaching a skilled person how to purify a protein heterologous expressed by host cells, are well known in the art.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilised. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind are known from the prior art.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucin-zipper domains. However, there are also strategies in which the interaction between the VH/VL regions of the scFv are used for the multimerisation (e.g. dia-, tri- and pentabodies). By diabody the skilled person means a bivalent homodimeric scFv derivative. The shortening of the Linker in an scFv molecule to 5-10 amino acids leads to the formation of homodimers in which an inter-chain VH/VL-superimposition takes place. Diabodies may additionally be stabilised by the incorporation of disulphide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a Linker region. Examples of minibody-antibody proteins are known from the prior art.

By triabody the skilled person means a: trivalent homotrimeric scFv derivative. ScFv derivatives wherein VH-VL are fused directly without a linker sequence lead to the formation of trimers.

By “scaffold proteins” a skilled person means any functional domain of a protein that is coupled by genetic cloning or by co-translational processes with another protein or part of a protein that has another function.

The skilled person will also be familiar with so-called miniantibodies which have a bi-, tri- or tetravalent structure and are derived from scFv. The multimerisation is carried out by di-, tri- or tetrameric coiled coil structures.

By definition any sequences or genes introduced into a host cell are called “heterologous sequences” or “heterologous genes” or “transgenes” with respect to the host cell, even if the introduced sequence or gene is identical to an endogenous sequence or gene in the host cell. A sequence is called “heterologous sequence” even when the sequence of interest is the endogenous sequence but the sequence has been (artificially/intentionally/experimentally) brought into the cell and is therefore expressed from a locus in the host genome which differs from the endogenous gene locus.

A sequence is called “heterologous sequence” even when the sequence (e.g. cDNA) of interest is the endogenous sequence but expression of this sequence is effected by an alteration/modification of a regulatory sequence, e.g. a promoter alteration or by any other means.

A “heterologous” protein is thus a protein expressed from a heterologous sequence.

Heterologous gene sequences can be introduced into a target cell by using an “expression vector”, preferably an eukaryotic, and even more preferably a mammalian expression vector. Methods used to construct vectors are well known to a person skilled in the art and described in various publications. In particular techniques for constructing suitable vectors, including a description of the functional components such as promoters, enhancers, termination and polyadenylation signals, selection markers, origins of replication, and splicing signals, are known in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif.

In a preferred embodiment the expression vector comprises at least one nucleic acid sequence which is a regulatory sequence necessary for transcription and translation of nucleotide sequences that encode for a peptide/polypeptide/protein of interest.

The term “expression” as used herein refers to transcription and/or translation of a heterologous nucleic acid sequence within a host cell. The level of expression of a desired product/protein of interest in a host cell may be determined on the basis of either the amount of corresponding mRNA that is present in the cell, or the amount of the desired polypeptide/protein of interest encoded by the selected sequence as in the present examples. For example, mRNA transcribed from a selected sequence can be quantitated by Northern blot hybridization, ribonuclease RNA protection, in situ hybridization to cellular RNA or by PCR. Proteins encoded by a selected sequence can be quantitated by various methods, e.g. by ELISA, by Western blotting, by radioimmunoassays, by immunoprecipitation, by assaying for the biological activity of the protein, by immunostaining of the protein followed by FACS analysis or by homogeneous time-resolved fluorescence (HTRF) assays.

“Increased expression” means at least 2-fold higher levels of the specific mRNA transcript compared to an untreated control cell. This applies equally for both secretion enhancing genes and anti-apoptotic genes.

The mRNA level in this assay can be detected either by northern blotting or quantitative/real-time RT-PCR using transcript-specific primers such as e.g. the XBP-1 specific primers having the SEQ ID NOs. 17 and 18 (see e.g. FIG. 9 and Example 11)

For a secretion enhancing gene the term “increasing the expression or activity” means at least 2-fold higher levels of the specific mRNA transcript compared to an untreated control cell and secretion of at least 20% more protein-of-interest compared to untransfected cells.

For an anti-apoptotic gene the term “increasing the expression or activity” means at least 2-fold higher levels of the specific mRNA transcript compared to an untreated control cell or, terms of activity, measurement of e.g. the proteolytic activation of the effector caspases-3 or -9, e.g. by detection of specific cleavage products in Western Blot experiments or measurement of DNA laddering which can be visualized in agarose gelelectrophoresis or AnnexinV-exposure on the cell surface, whereby decreased measurement values in these assay indicate increased activity of the anti-apoptotic gene.

“Transfection” of eukaryotic host cells with a polynucleotide or expression vector, resulting in genetically modified cells or transgenic cells, can be performed by any method well known in the art. Transfection methods include but are not limited to liposome-mediated transfection, calcium phosphate co-precipitation, electroporation, polycation (such as DEAE-dextran)-mediated transfection, protoplast fusion, viral infections and microinjection. Preferably, the transfection is a stable transfection. The transfection method that provides optimal transfection frequency and expression of the heterologous genes in the particular host cell line and type is favoured. Suitable methods can be determined by routine procedures. For stable transfectants the constructs are either integrated into the host cell's genome or an artificial chromosome/mini-chromosome or located episomally so as to be stably maintained within the host cell.

The practice of the present invention will employ, unless otherwise indicated, conventional techniques of cell biology, molecular biology, cell culture, immunology and the like which are in the skill of one in the art. These techniques are fully disclosed in the current literature.

The invention relates to a method of producing a heterologous protein of interest in a cell comprising increasing the expression or activity of a secretion enhancing gene, and increasing the expression or activity of an anti-apoptotic gene, and effecting the expression of said protein of interest, whereby the secretion enhancing gene is a gene encoding a protein whose expression or activity is induced during one of the following cellular processes: plasma-cell differentiation, unfolded protein response (UPR), endoplasmatic reticulum overload response (EOR).

The invention relates to a method of producing a heterologous protein of interest in a cell comprising increasing the expression or activity of a secretion enhancing gene, and increasing the expression or activity of an anti-apoptotic gene, and effecting the expression of said protein of interest, whereby the secretion enhancing gene confers a growth and/or survival disadvantage to said cell.

The invention furthermore relates to a method of producing a heterologous protein of interest in a cell comprising increasing the expression or activity of a secretion enhancing gene, and increasing the expression or activity of an anti-apoptotic gene, and expressing said protein of interest, whereby the secretion enhancing gene confers a growth and/or survival disadvantage to said cell.

In a specific embodiment of the present invention the method is characterized in that the cell has at least 2-fold higher expression levels of the specific mRNA transcript of the secretion enhancing gene in comparison to an untreated control cell and the cell secretes at least 20% more protein-of-interest compared to untransfected cells, and the cell has at least 2-fold higher expression levels of the specific mRNA transcript of the anti-apoptotic-gene in comparison to an untreated control cell.

Furthermore, increased activity of the anti-apoptotic gene can be measured by decreased measurement values in assays as described in the present invention (e.g. detection of specific cleavage products in Western Blot experiments or measurement of DNA laddering which can be visualized in agarose gelelectrophoresis or AnnexinV-exposure on the cell surface).

In a specific embodiment of the present invention the method is characterized in that the secretion enhancing gene is the X-box binding protein-1 (XBP-1) or a derivative thereof including all XBP-1 splice variants as well as all XBP-1 mutants.

In a preferred embodiment of the present invention the method is characterized in that the XBP-1 expression level is at least 2-fold higher in comparison to an untreated control cell as measurable by real time PCR using the primers having SEQ ID NOs 17 and 18.

In further specific embodiment of the present invention the method is characterized in that the secretion enhancing gene encodes a XBP-1 protein as defined by SEQ ID NO:2.

In another specific embodiment of the present invention the method is characterized in that the secretion enhancing gene is a gene encoding a protein which directly induces the expression or activity of X-box binding protein-1 (XBP-1). Such gene is preferably IRE, ATF4 (also known as CREB2, TXREB, CREB-2 or TAX Responsive Element B67 (TAXREB67)), ATF6 or IRF4.

In a further embodiment of the present invention the method is characterized in that the secretion enhancing gene is a gene whose promoter comprises one or more ER-stress responsive elements (ERSE) as defined by SEQ ID NO:9 or SEQ ID NO:10 or one or more unfolded protein response elements (UPRE) as defined by SEQ ID NO:11 or SEQ ID NO:12, and whereby said gene is preferably an XBP-1 target gene.

In a further specific embodiment of the present invention the method is characterized in that the anti-apoptotic gene is a gene encoding a protein which inhibits or delays the activation of the effector caspases 3 and/or 9.

In another embodiment of the present invention the method is characterized in that the anti-apoptotic gene is a protein belonging to the inhibitor of apoptosis (IAP) family of proteins which is characterized by one or more copies of an amino acid motive termed BIR (baculovirus IAP repeat) domain.

In another specific embodiment of the present invention the method is characterized in that the anti-apoptotic gene comprises a BIR consensus sequence (SEQ ID NO:13) or a derivative thereof.

In a preferred embodiment of the present invention the method is characterized in that the anti-apoptotic gene is a gene encoding XIAP (SEQ ID NO:4) or a derivative or mutant thereof.

In another preferred embodiment of the present invention the method is characterized in that the anti-apoptotic gene is a protein belonging to the Bcl-2 family of proteins which is characterized by its Bcl-2 homology (BH) domains.

In a specific embodiment of the present invention the method is characterized in that the anti-apoptotic gene comprises a Bcl-2 consensus sequence (SEQ ID NO:14) or a derivative thereof.

In another specific embodiment of the present invention the method is characterized in that the anti-apoptotic gene is a gene encoding Bcl-XL (SEQ ID NO:6) or a derivative thereof. In a specific embodiment of the present invention the method is characterized in that the anti-apoptotic gene is a gene encoding Bcl-XL mutant (SEQ ID NO:8) or a derivative thereof.

In a further embodiment of the present invention the method is characterized in that said method results in increased specific cellular productivity and/or titer of said protein of interest in said cell in comparison to a control cell expressing said protein of interest, but whereby said control cell does not have increased expression or activity of a secretion enhancing protein and an anti-apoptotic protein.

In a further specific embodiment of the present invention the method is characterized in that the increase in productivity is about 5% to about 10%, about 11% to about 20%, about 21% to about 30%, about 31% to about 40%, about 41% to about 50%, about 51% to about 60%, about 61% to about 70%, about 71% to about 80%, about 81% to about 90%, about 91% to about 100%, about 101% to about 149%, about 150% to about 199%, about 200% to about 299%, about 300% to about 499%, or about 500% to about 1000%.

In an embodiment of the present invention the method is characterized in that said cell is a eukaryotic cell such as a yeast, plant, worm, insect, avian, fish, reptile or mammalian cell. In a preferred embodiment said avian cell is a chicken or duck cell line.

In a further preferred embodiment said eukaryotic cell is a mammalian cell selected from the group consisting of a Chinese Hamster Ovary (CHO) cell, monkey kidney CV 1 cell, monkey kidney COS cell, human lens epitheliaim PER.C6™ cell, human embryonic kidney cell, human amniocyte cell, human myeloma cell, HEK293 cell, baby hamster kidney cell, African green monkey kidney cell, human cervical carcinoma cell, canine kidney cell, buffalo rat liver cell, human lung cell, human liver cell, mouse mammary tumor or myeloma cell, a dog, pig, macaque, rat, rabbit, cat and goat cell.

In a most preferred embodiment said CHO cell is CHO wild type, CHO K1, CHO DG44, CHO DUKX-B11, CHO Pro-5, preferably CHO DG44.

In a specific embodiment of the present invention the method is characterized in that the protein of interest is a membrane or secreted protein.

In a preferred embodiment the protein of interest is an antibody or antibody fragment.

In a further preferred embodiment the antibody is monoclonal, polyclonal, mammalian, murine, chimeric, humanized, primatized, primate, human or an antibody fragment or derivative thereof such as antibody, immunoglobulin light chain, immunoglobulin heavy chain, immunoglobulin light and heavy chains, Fab, F(ab')2, Fc, Fc-Fc fusion proteins, Fv, single chain Fv, single domain Fv, tetravalent single chain Fv, disulfide-linked Fv, domain deleted, minibody, diabody, or a fusion polypeptide of one of the above fragments with another peptide or polypeptide, Fc-peptide fusion, Fc-toxine fusion, scaffold proteins.

The invention further relates to a method of increasing specific cellular productivity of a membrane or secreted protein of interest in a cell comprising introducing into a cell one or more vector systems comprising nucleic acid sequences encoding at least three polypeptides whereby a first polynucleotide encodes a protein having secretion enhancing activity and a second polynucleotide encodes a protein having anti-apoptotic activity and a third polynucleotide encodes a protein of interest and whereby the protein of interest and the protein having secretion enhancing activity and the protein having anti-apoptotic activity are expressed by said cell and whereby the secretion enhancing gene is a gene encoding a protein whose expression or activity is induced during one of the following cellular processes: plasma-cell differentiation, unfolded protein response (UPR), endoplasmatic reticulum overload response (EOR).

In another embodiment said method is characterized in that the secretion enhancing gene confers a growth and/or survival disadvantage to said cell.

In a specific embodiment of the present invention said method is characterized in that the vector systems or said polynucleotides are introduced simultaneously. In another specific embodiment of the present invention the method is characterized in that the vector systems or said polynucleotides are introduced sequentially.

In another specific embodiment of the present invention said method is characterized in that the vector systems are mono-, bi-, or tri-cistronic.

In a further specific embodiment of the inventive method said secretion enhancing gene and said anti-apoptotic gene are introduced into a cell already containing a gene/protein of interest.

In an additional embodiment of the present invention said method is characterized in that the method comprises an amplification step of one or all transgenes.

In another additional embodiment of the present invention said method is characterized in that the method does not comprise an amplification step of one or all transgenes.

The invention further relates to an expression vector comprising two polynucleotides, a first polynucleotide encoding for a protein having secretion engineering activity and a second polynucleotide encoding for a protein having anti-apoptosis activity and a third polynucleotide encoding for a protein of interest, whereby the secretion enhancing gene is a gene encoding a protein whose expression or activity is induced during one of the following cellular processes: plasma-cell differentiation, unfolded protein response (UPR), endoplasmatic reticulum overload response (EOR).

In a preferred embodiment the secretion enhancing gene is a gene which confers a growth and/or survival disadvantage to said cell.

In a preferred embodiment the expression vector comprises a gene encoding for XBP-1. In a further preferred embodiment the expression vector comprises a gene encoding for XIAP or Bcl_Xl mutant.

In a most preferred embodiment the expression vector comprises a gene encoding for XBP-1 and another gene encoding for XIAP or Bcl_Xl mutant. Most preferred is the combination of XBP-1 and XIAP.

The invention further relates to a method of generating a cell comprising introducing into a cell one or more vector systems comprising nucleic acid sequences encoding at least three polypeptides whereby
a first nucleic acid sequences encodes a protein having secretion enhancing activity and
a second nucleic acid sequences encodes a protein having anti-apoptotic activity and
a third nucleic acid sequences encodes a protein of interest and
whereby the protein of interest and the protein having secretion enhancing activity and the protein having anti-apoptotic activity are expressed by said cell and
whereby the secretion enhancing gene is a gene encoding a protein whose expression or activity is induced during one of the following cellular processes: plasma-cell differentiation, unfolded protein response (UPR), endoplasmic reticulum overload response (EOR).

In a preferred embodiment of said method the nucleic acid sequence encoding a protein having secretion enhancing activity is XBP-1.

In another preferred embodiment the nucleic acid sequence encoding a protein having anti-apoptotic activity is XIAP or a member of the BCL-2 family, preferably BCL-2 or BCL-XL. XIAP is particularly preferred.

The invention further relates to a cell generated according to any of the inventive methods. The invention furthermore relates to a cell comprising the expression vector of the present invention.

In a specific embodiment said secretion enhancing gene is a gene encoding a protein whose expression or activity is induced during one of the following cellular processes: plasma-cell differentiation, unfolded protein response (UPR), endoplasmatic reticulum overload response (EOR).

In a further specific embodiment the cell expresses at least three heterologous genes: a secretion enhancing gene, which confers a growth and/or survival disadvantage to said cell, an anti-apoptotic gene, and a protein of interest.

In a preferred embodiment the secretion enhancing gene is XBP-1.

In another preferred embodiment the anti-apoptotic gene is XIAP or a member of the BCL-2 family, preferably BCL-2 or BCL-XL.

In another embodiment of the present invention said cell is characterized in that said cell is a eukaryotic cell such as a yeast, plant, worm, insect, avian, fish, reptile or mammalian cell. Preferably said avian cell is a chicken or duck cell line.

In a preferred embodiment said cell is a mammalian cell selected from the group consisting of a Chinese Hamster Ovary (CHO) cell, monkey kidney CV1 cell, monkey kidney COS cell, human lens epithelium PER.C6™ cell, human embryonic kidney HEK293 cell, human amniocyte cell, human myeloma cell, baby hamster kidney cell, African green monkey kidney cell, human cervical carcinoma cell, canine kidney cell, buffalo rat liver cell, human lung cell, human liver cell, mouse mammary tumor or myeloma cell such as NS0, a dog, pig, macaque, rat, rabbit, cat and goat cell.

In a further preferred embodiment said CHO cell is CHO wild type, CHO K1, CHO DG44, CHO DUKX-B11, CHO Pro-5, preferably CHO DG44.

The invention furthermore relates to a use of a protein having secretion enhancing activity in combination with a protein having anti-apoptotic activity to increase production of a protein of interest in vitro, whereby the secretion enhancing gene is a gene encoding a protein whose expression or activity is induced during one of the following cellular processes: plasma-cell differentiation, unfolded protein response (UPR), endoplasmic reticulum overload response (EOR).

The invention additionally relates to a use of a protein having secretion enhancing activity in combination with a protein having anti-apoptotic activity to increase production of a protein of interest in vitro, whereby the secretion enhancing gene confers a growth and/or survival disadvantage to said cell.

In preferred specific embodiments such use is for biopharmaceutical manufacturing, diagnostic applications or for research and development purposes.

The invention generally described above will be more readily understood by reference to the following examples, which are hereby included merely for the purpose of illustration of certain embodiments of the present invention. The following examples are not limiting. They merely show possible embodiments of the invention. A person skilled in the art could easily adjust the conditions to apply it to other embodiments.

### Experimental

### Materials and Methods

### Cell Culture

### a) Adherent Cultures

CHO-K1 cells are maintained as monolayer in F12-Media (Gibco) supplemented with 5% FCS (Biological Industries). The cells are incubated in surface-aerated T-flasks (Nunc) in humidified incubators (Thermo) with 5% CO₂ at 37° C. Cultures are split by trypsination and re-seeding twice a week. The seeding density is typically 3-6×10⁴ cells/cm², allowing the cells to reach confluency in 3-4 days.

### b) Suspension Cultures

Suspension cultures of mAB producing CHO-DG44 cells (Urlaub et al., 1986) and stable transfectants thereof are incubated in a BI proprietary chemically defined, serum-free media. Seed stock cultures are sub-cultivated every 2-3 days with seeding densities of 3×10⁵-2×10⁵ cells/mL respectively. Cells are grown in T-flasks or shake flasks (Nunc). T-flasks are incubated in humidified incubators (Thermo) and shake flasks in Multitron HT incubators (Infors) at 5% CO₂, 37° C. and 120 rpm.

The cell concentration and viability is determined by trypan blue exclusion using a hemocytometer.

### Expression Vectors

To generate pBIP-XBP1, pCDNA3-XBP-1(s), containing the spliced variant of human X-box-binding protein, is XbaI digested and blunted using Klenow enzyme. A second digestion is performed using HindIII. The fragment is then cloned into pBIP (BI proprietary) which is BsrGI (blunt) and HindIII digested (all enzymes are obtained from New England Biolabs). For selection of stable cells the pBIP vector contains a puromycin resistance cassette. The expression of the heterologous gene is driven by a CMV promoter/enhancer combination.

For the generation of the bicicstronic vectors pIRES (Clonetech) is NotI digested and blunted using Klenow enzyme. The resulting linearized vector is then EcoRI digested to yield a IRES containing fragment. This fragment is cloned into pBIP which is BsrGI and EcoRI digested to yield pBIP-IRES. To generate the further expression constructs the following genes are used:

| | | Cut with | In Cistron | |
|---|---|---|---|---|
| Gene | Donor Plasmid | Enzyme(s) | inserted | Final Vector |
| XIAP | pEBiP-XIAP | XhoI/EcoRI | First | pBIP-IRES-XIAP |
| BclxL₍₄₆₋₈₃₎ | pBIG4 | EcoRI | First | pBIP-IRES-BclxL₍₄₆₋₈₃₎ |
| XBP1 | pCDNA3-XBP1, | XbaI | Second of: | pBIP-IRES-XBP1 |
| | (PCR amplification) | | pBIP-IRES | pBIP-IRES-XIAP-XBP1 |
| | | | pBIP-IRES-XIAP | pBIP-IRES- BclxL₍₄₆₋₈₃₎- |
| | | | pBIP-IRES-BclxL₍₄₆₋₈₃₎ | XBP1 |

The resulting vectors have a constant layout with the anti-apoptotic protein (e.g. XIAP) in the first expression cistron and the secretion enhancing protein (e.g. XBP1) in the second cistron.

### Generation of Stable Monoclonal CHO Cell Lines

All cells are transfected in 6-well plates using Lipofectamine™ and Plus™ reagent (Invitrogen) according to the manufacturer's protocol. For the generation of stable populations, the antibiotic puromycin is added 48 h after transfection at a concentration of 10 mg/L. Cells are cultivated in static cultures until growth is observed by microscopic inspection and than subjected to seedstock cultivation in chemically defined BI proprietary medium.

Clones are generated by single cell cloning in 96-well plates using a fluorescent activated cell sorter (FACS) from Beckman Coulter (Ecpics Altra HyPersort System).

### Western Blot

For nuclear extracts 5×10⁶ cells/mL are pelleted by centrifugation for 5 min at 200 g and washed in ice cold PBS. Pellet is resuspended in 250 μl NP40-buffer (0.5% NP40, 10 mM HEPES pH 7.9, 10 mM KCl, 1 mM EDTA, 40 μL/mL Complete™ (Roche)) and incubated 5 min on ice. Nuclei were spun down for 5 min at 800 g. The pellet is washed in 500 μL CE-buffer (10 mM HEPES pH 7.9, 10 mM KCl, 1 mM EDTA, 40 μL/mL Complete) and nuclei are then resuspended in 250 μL NE-buffer (250 mM Tris pH 7.8, 60 mM KCl, 1 mM EDTA, 40 μL/mL Complete) and broken up with 3 freeze-thaw cycles (liquid nitrogen and 37° C. water bath). Debris is pelleted for 10 min at 16000 g and supernatant further analysed.

For whole cell lysates 5×10⁶ cells/mL are pelleted by centrifugation for 5 min at 200 g, washed in ice cold PBS and resuspended in lysis buffer (1% NP40, 50 mM HEPES pH 7.4, 150 mM NaCl, 25 mM NaF, 1 mM EDTA, 5 mM EGTA, 40 μL/mL Complete™ (Roche)) and incubated for 15 min on ice. Cell debris is pelleted for 10 min at 16000 g and supernatant further analysed.

For Western blot analysis equal volumes of nuclear extracts or equal amount of protein for whole cell lysates are separated with MOPS buffer on a NuPAGE 10% Bis-Tris-Gel (Invitrogen) according to the manufacturer's protocol. The proteins are transferred on a PVDF membrane (Millipore) using transfer buffer in XCell II blot module (Invitrogen). Blocking is done for 1 h at room temperature with blocking agent (Invitrogen). Rabbit anti-XBP-1 (Biolegend) is used as primary antibody in 1:1000 dilution. The secondary antibody is goat anti-rabbit IgG (H+L) HRP Conjugate (BioRad) in 1:10000 dilution. For detection the ECL Plus system (Amersham Pharmacia) is used.

### Fed Batch Cultivation

Cells are seeded at 3×10⁵ cells/ml into 1000 ml shake flasks in 250 ml of BI-proprietary production medium without antibiotics or MTX (Sigma-Aldrich, Germany). The cultures are agitated at 120 rpm in 37° C. and 5% CO₂ which is later reduced to 2% as cell numbers increase. Culture parameters including pH, glucose and lactate concentrations are determined daily and pH is adjusted to pH 7.0 using NaCO₃ as needed. BI-proprietary feed solution is added every 24 hrs. Cell densities and viability are determined by trypan-blue exclusion using an automated CEDEX cell quantification system (Innovatis AG, Bielefeld, Germany). Samples from the cell culture fluid are collected at and subjected to titer measurement by ELISA.

For ELISA antibodies against human-Fc fragment (Jackson Immuno Research Laboratories) and human kappa light chain HRP conjugated (Sigma) are used.

Cumulative specific productivity is calculated as product concentration at the given day divided by the “integral of viable cells” (IVC) until that time point.

### Colony Formation Assay (CFA)

CHO-K1 cells are trypsinated 24 h after transfection. 1×10⁵ cells are transferred to a 9 cm Petri dish containing finally 12 ml fresh culture medium. The cells are allowed to adhere for 24 h under culture conditions when the selection antibiotic puromycin is added in a final concentration of 15 mg/L. The dishes are cultured at 37° C. and 5% CO₂ atmosphere for 12 days when the colonies are fixed with ice cold Aceton/Methanol (1:1) for five minutes. The fixed colonies are then stained with Giemsa (1:20 in dest. Water) for 15 minutes. To remove excess dye the plates are washed with dest. water and air dried. Colonies are counted manually for analysis.

### Antibody Productivity

### a) ELISA

Antibody producing CHO-DG44 are transfected with bicistronic vectors to analyse the effect of heterologous protein expression on mAb productivity. To assess the productivity in seed stock culture, samples from cell culture supernatant are collected from three consecutive passages. The product concentration is then analysed by enzyme linked immunosorbent assay (ELISA). For ELISA antibodies against human-Fc fragment (Jackson Immuno Research Laboratories) and human kappa light chain HRP conjugated (Sigma) are used. Together with the cell densities and viabilities the specific productivity can be calculated as follows:

$$qp = \frac{\frac{( {mAb}_{P + 1} + {mAb}_{P} )}{2}}{( {t}_{P + 1} - {t}_{P} ) * ( \frac{{cc}_{P + 1} + {cc}_{P}}{2} )}$$

qp=specific productivity (pg/cell/day)
mAb=antibody concentration (mg/L)
t=time point (days)
cc=cell count (×10⁶ cells/mL)

### P=Passage

### b) HTRF-Assay

To evaluate the product concentration of monoclonal colonies in 96 well plates a sample of supernatant is analysed using the homogeneous time resolved fluorescence resonance (HTRF®) technique (CISBIO). The colony size is classified by microscopic inspection in large and medium colonies. Supernatant collected from wells with monoclonal colonies is incubated with an anti-FC donor antibody (crytate labeled) and an Anti-kappa light chain acceptor antibody (D2-dye labeled) for 1 h at room temperature to detect the secreted antibody product. In case that donor and acceptor have bound to the target antibody, the fluorescence resonance energy transfer principle (FRET) can be applied by exitation of the donor at 337 nm. This leads to an energy transfer to the acceptor who emits light at 665 nm. This light emission at 665 nm correlates with the amount of antibody present in the sample and was measured using an Ultra Evolution Reader (Tecan).

### Apoptosis Assay

Apoptosis is detected using the Annexin V-FITC Kit I (BD Biosciences, Erembodegem, Belgium) according to the manufacturer's protocol. Equal cell numbers are washed with PBS and resuspended in binding buffer. For staining, 100 μL of the cell suspension is transferred to a new reaction tube and 5 μL of an Annexin V conjugate followed by 2 μL of propidium iodide (PI) for counterstaining are added. After an incubation period of 20 min in the dark, the cells are resuspended in 400 μL of PBS and analyzed by flow cytometry (Beckmann Coulter, ex./em. wavelength for FITC 488/524 nm and for PI 488/620 nm).

### Real-Time PCR

Quantitative real-time PCR is used for quantification of specific XBP-1 mRNA transcript levels, using the SYBR® Green Mastermix Kit (Applied Biosystems, Foster City, USA). All samples are prepared in triplicates and qPCR is performed in an iCycler iQ5 (BioRad, Hercules, USA) according to the manufacturer's protocol. The annealing temperature is 58° C. and data are collected at the end of every 72° C. extension cycle. Beta-tubulin levels are used for standardization.

The following oligonucleotides are used as PCR primers:

| | |
|---|---|
| Tub_for: | |
| 5′-CTCAACGCCGACCTGCGCAAG-3′, | (SEQ ID NO: 15) |
| Tub_rev: | |
| 5′-ACTCGCTGGTGTACCAGTGC-3′, | (SEQ ID NO: 16) |
| XBP1_for: | |
| 5′-TGGTTGAGAACCAGGAGTTA-3′, | (SEQ ID NO: 17) |
| XBP1_rev: | |
| 5′-GCTTCCAGCTTGGCTGATG-3′, | (SEQ ID NO: 18) |

### EXAMPLES

### Example 1

### Correlation of XBP-1 Expression Level and Productivity

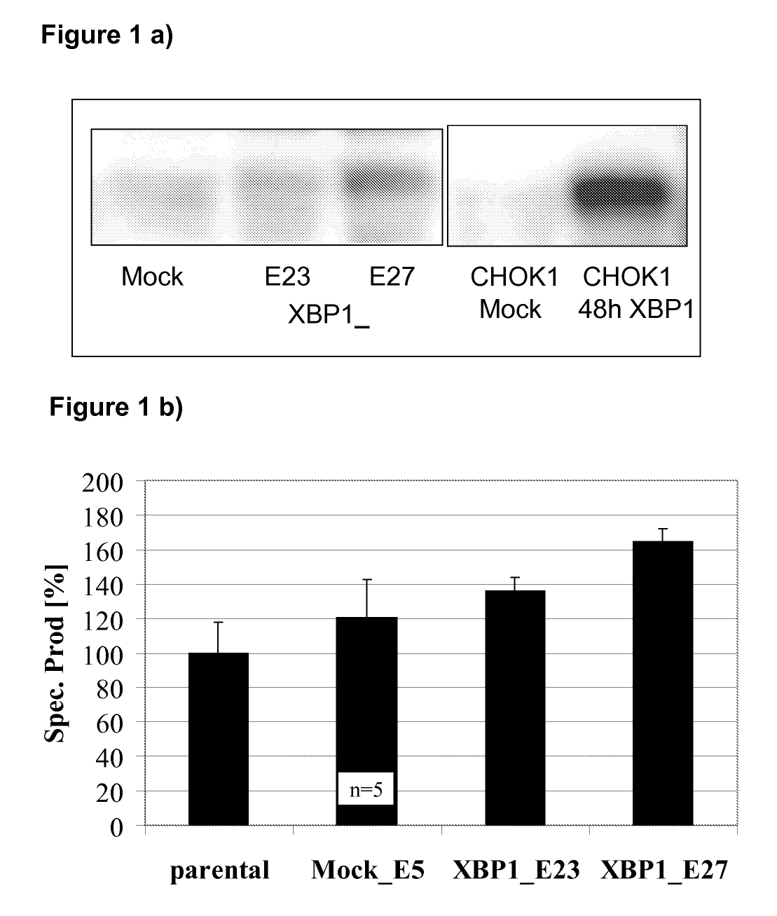

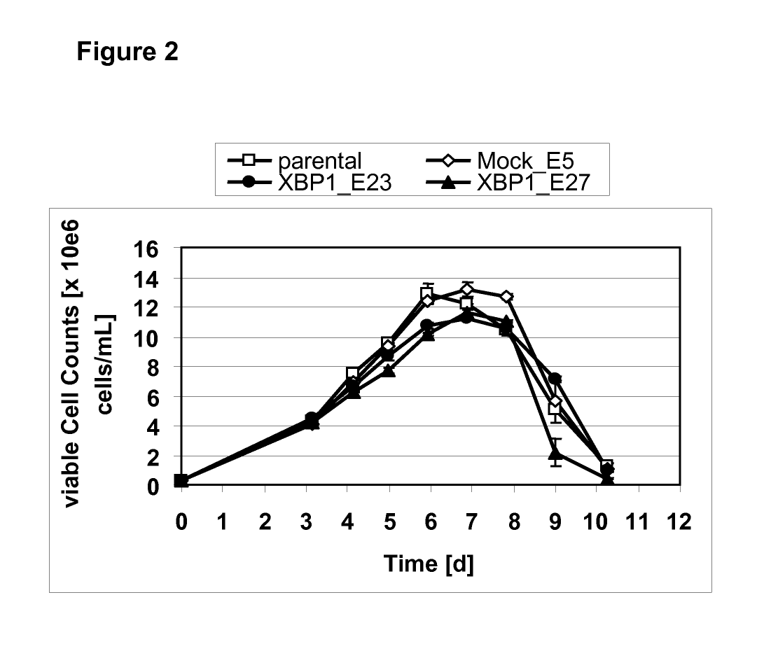

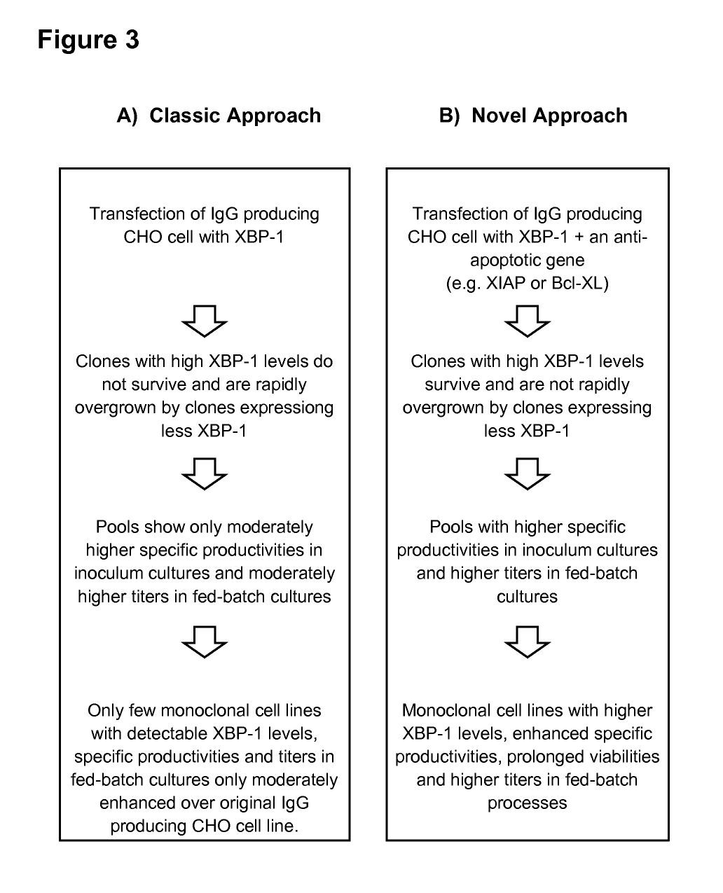

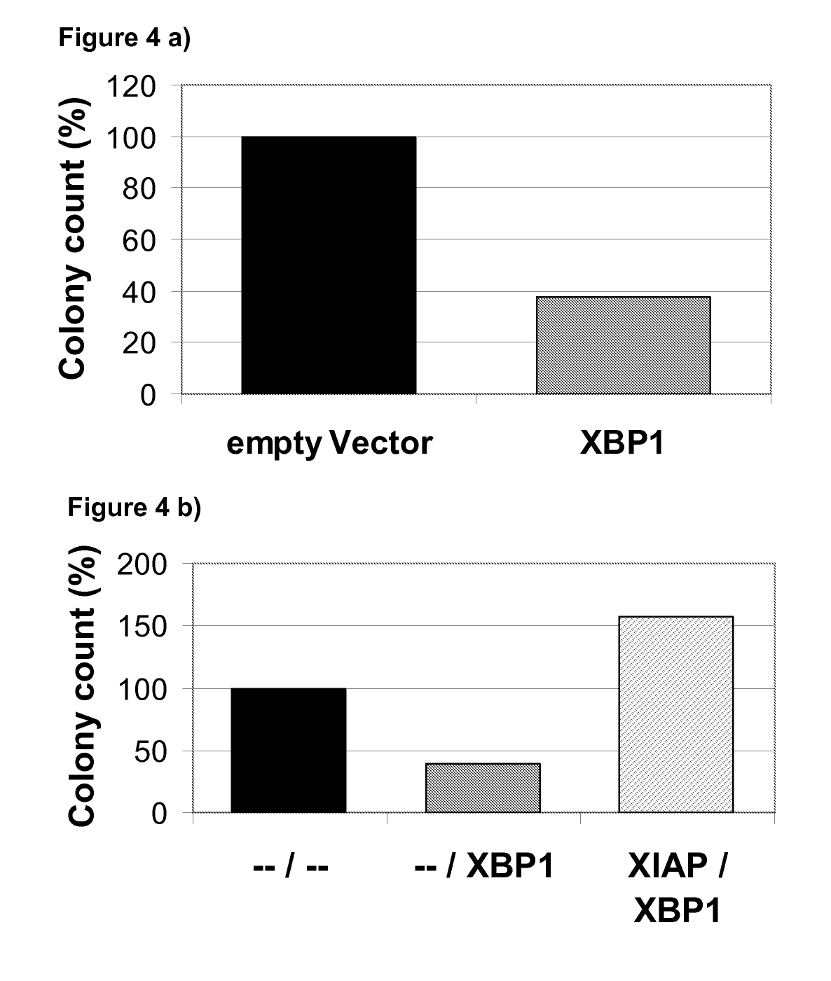

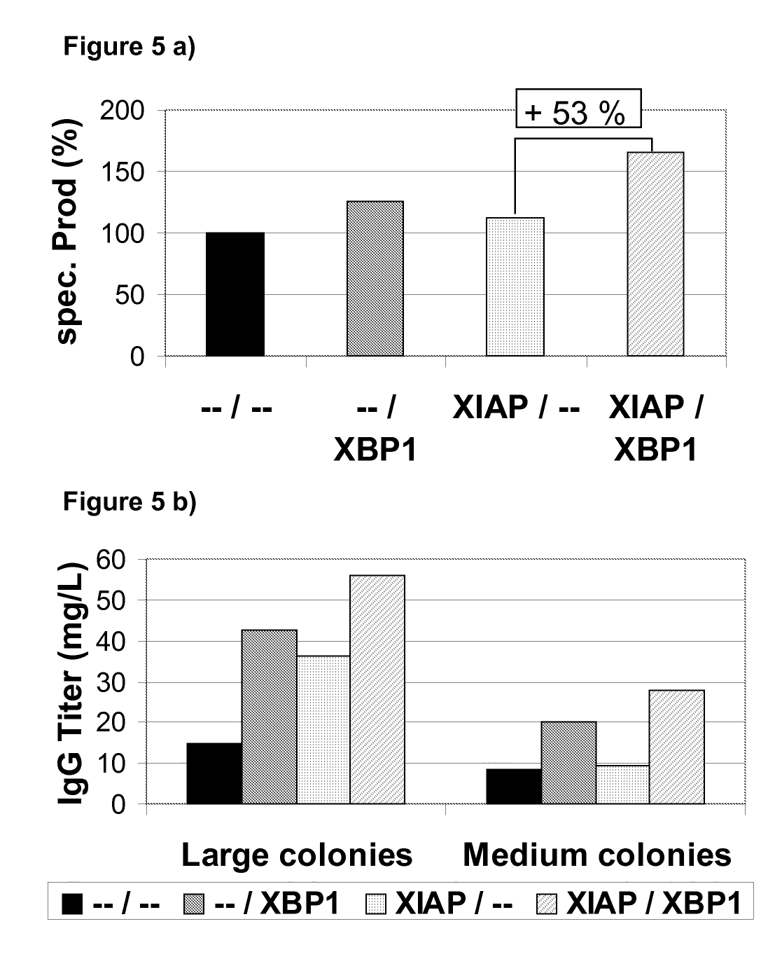

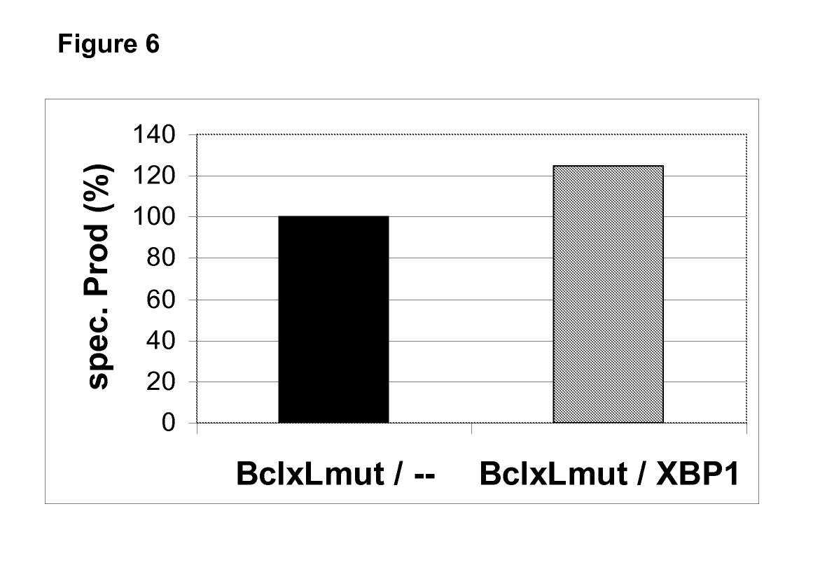

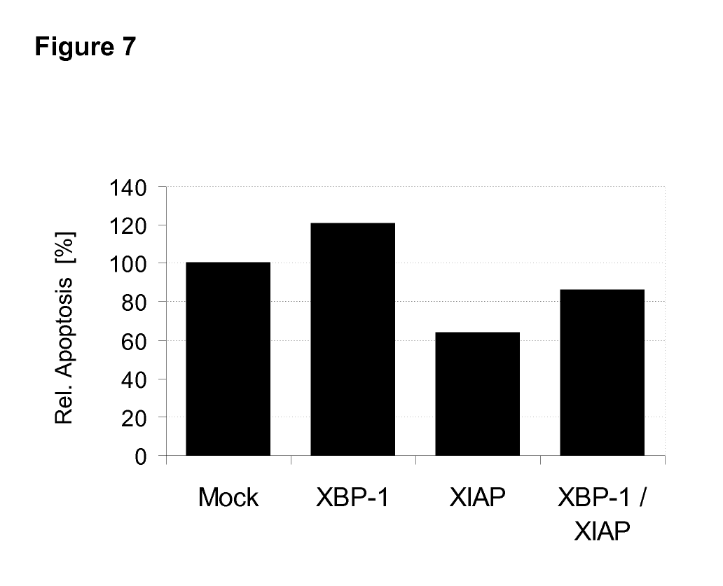

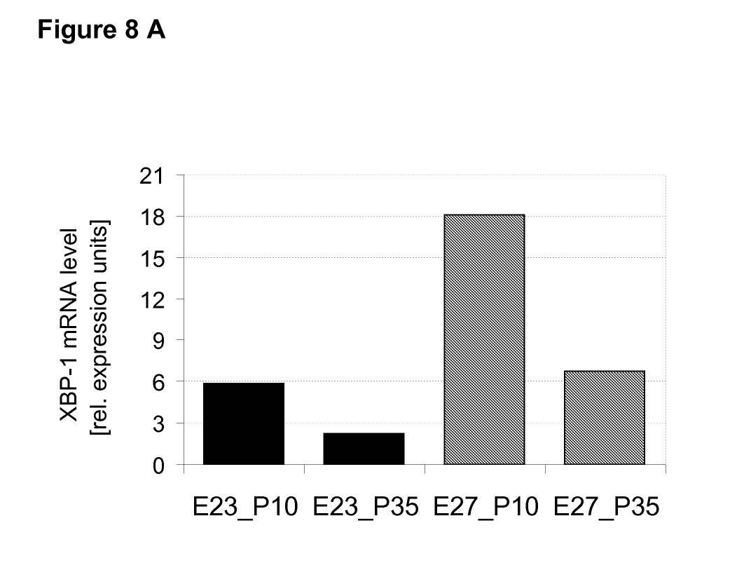

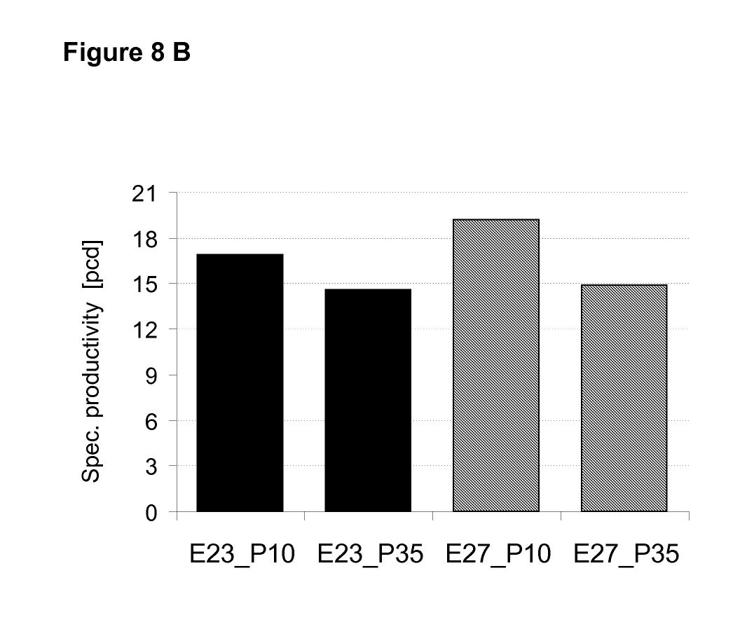

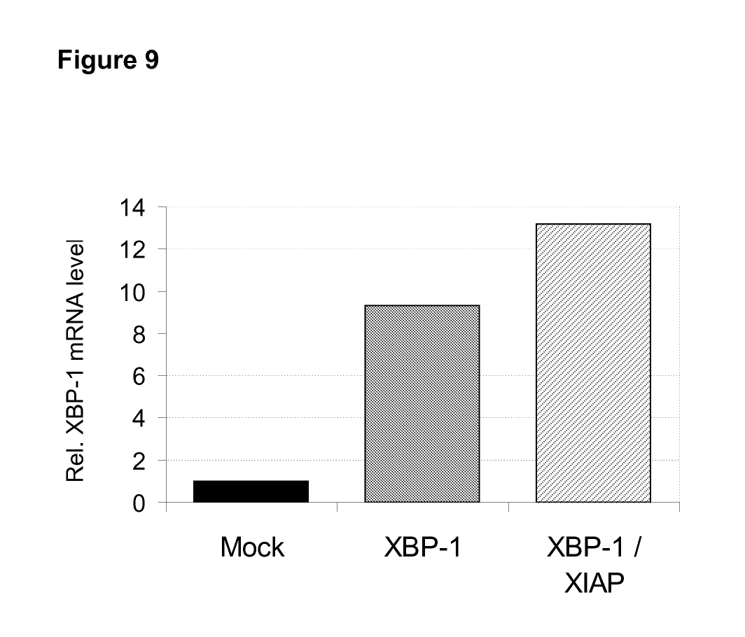

A CHO-DG44 cell line expressing a therapeutic IgG molecule (“parental”) is stably transfected with a plasmid encoding XBP-1(s) or an empty plasmid (“Mock”) control. XBP-1(s) transgene expression in monoclonal cell lines is analysed by Western Blot using lysates from transient mock and XBP-1(s) transfections in CHO-K1 cells as negative and positive control, respectively. Out of 14 XBP-1 transfected clones, the two cell lines XBP1_E23 and XBP1_E27 show the lowest and highest XBP-1(s) expression respectively (FIG. 1a) and are therefore selected for further analysis. For a stringent control of the significance of any effect of expression of XBP-1 on productivity, 5 mock clones are also screened and the cell line with the highest specific productivity is selected for all further experiments (Mock_E5). All cell lines are than cultivated according to a 2d-2d-3d rhythm that is typically used in industrial inoculum schemes for large scale manufacturing. Cell culture supernatants are collected over 5 to 11 passages during cell passaging and analyzed for antibody concentration by IgG-ELISA. Viable cell counts for each passage are then used to calculate the average specific productivities of the cell lines.

As shown in FIG. 1b, the specific productivity of the cells expressing XBP-1(s) is enhanced up to 60% when compared to the parental cell line. Notably, this effect is more pronounced in clone XBP-1_E27, which exhibited higher XBP-1 expression, whereas it is less significant in clone E23, which shows only a weak XBP-1 signal in the Western Blot. This indicates that there is a positive correlation between the level of XBP-1 expression and specific productivity.

### Example 2

### Heterologous XBP-1 Expression Leads to Reduced Growth in Fed-Batch Processes

To test if the increased specific productivity during serial cultivation translates into higher antibody yield in a production process, the monoclonal cell lines described in Example 1 (parental, mock_E5, XBP1_E23 and XBP1_E27) are analysed in a scale-down fed-batch process format. Shake flasks are inoculated at a seeding density of 0.25×10⁶ cells/mL and cultivated for 10 days with daily feeding and pH adjustment to closely simulate controlled bioreactor conditions.

As seen in FIG. 2, parental and mock cell lines show an almost identical growth profile. Peak cell densities reached are around 13×10⁶ viable cells/mL for both cell lines. In comparison, XBP-1(s) expressing cell lines grow slower which becomes apparent already at day 5 and in addition reach lower maximal cell densities of about 11×10⁶ viable cells/mL. Together, the growth reduction seen in XBP-1 expressing cell clones results in lower IVC's over time which in a production process translates into a reduced overall product yield.

### Example 3

### Heterologous Expression of XBP-1 Results in Reduced Cell Survival in Colony Formation Assays (CFA)

To quantitatively analyse whether forced expression of XBP-1 bears the risk of increasing the cell's sensitivity towards apoptosis, we make use of the colony cormation assay (CFA), a model system to study cell growth and survival.

Adherent CHO-K1 cells are transfected either with empty vectors (“mock”) or expression constructs the active, spliced form of human XBP-1, XBP-1(s). After 48 h, the cells are seeded into 10 cm-dishes and subjected to selection using the respective antibiotic, in this case puromycin. Under these conditions, most of the cells die and only those survive which have the expression plasmids stably integrated into their genomes. Following a recovery phase, these cells start to proliferate and grow out to colonies which after 10-14 days are fixed, stained with Giemsa and counted.

As seen in FIG. 4a, heterologous expression of XBP-1 results in a clear decrease in the number of cell colonies compared to the mock control, indicating that XBP-1 containing cells have a survival disadvantage.

The same results are obtained with bi-cistronic expression constructs where XBP-1 is contained in the second cistron FIG. 4b. However, when we co-express the X-linked inhibitor of apoptosis (XIAP) by cloning this gene into the first cistron in front of XBP-1 into the bi-cistronic expression cassette, we can completely restore colony counts. This demonstrates that reduced colony numbers obtained with XBP-1 transfected cells indeed can be attributed to increased apoptosis and this phenotype can be rescued by combined overexpression of an apoptosis inhibitor such as XIAP.

### Example 4

### Co-Expression of XBP-1 and XIAP Results in Increased Specific Productivities

To test our hypothesis, that co-expression of an anti-apoptotic gene facilitates the survival of XBP-1 expressing cells with enhanced secretory capacity, we analyse the effect of combined introduction of XBP-1 and XIAP on the specific productivity.

For this purpose, a well characterized CHO-derived monoclonal cell line producing IgG-type human antibody is stably transfected with a construct for bi-cistronic expression of two transgenes. The producer cells are transfected with either the empty vector as control, the same plasmid containing XBP-1 or XIAP alone or the construct expressing both transgenes simultaneously. The newly generated stable cell pools are than subjected to serial cultivation in shake flasks and split every two to three days. At the end of each passage, the cells are counted, cell culture supernatants are collected and the antibody titer is determined by ELISA. From these data, the specific productivity in pg per cell and day is calculated for each genotype.

As shown in FIG. 5A, heterologous expression XBP-1 alone in IgG producing cells already leads to an increase in the specific antibody productivity, whereas introduction of XIAP alone has only a minor effect. However, upon combined expression of both, XBP-1 and XIAP together, the specific productivity is increased by over 60% compared to control cells and over 50% in comparison to cells expressing only XIAP. Moreover, even the secretion enhancing effect of XBP-1 on the IgG producer cell line can be further increased by co-expression of the anti-apoptotic protein XIAP.

To elucidate the full potential of this multigene-engineering approach, the cell pools described above are then subjected to single-cell cloning to obtain homogenous monoclonal cell populations. Cells of each genotype are depositioned in 96-well plates with one single cell per well and after 1-3 weeks, the growing colonies are categorized according to size and medium samples are taken from each well and subjected to titer determination (FIG. 5B).

Already in the 96-well culture format, the results of the IgG titer measurement clearly reproduce the data obtained from stable cell pools. Importantly, the positive effect of XBP-1 and XIAP on antibody secretion which is seen in heterogenous cell pools is even more pronounced on the level of monoclonal cell lines, even though the exact viable cell numbers are not taken into account at this stage.

Taken together, these results demonstrate an additive, in some cases even a synergistic effect of the secretion-enhancing gene XBP-1 and the caspase inhibitor XIAP on the specific productivity of antibody producing cell line. Thus, these data represent the proof-of-concept for the multi-gene engineering approach to simultaneously target UPR/secretion and the pathway of regulated cell death.

### Example 5

### Enhanced Specific Productivities by Combining XBP-1 with Anti-Apoptotisis Engineering

To address the question whether the observed increase in titer and specific productivity upon combined expression of XBP-1 together with an anti-apoptotic gene is specific for XIAP, we test whether we can also achieve this goal by combining XBP-1 with other genes with anti-apoptotic function. For this purpose, IgG cells secreting a monoclonal human IgG antibody are transfected with either a Bcl-XL variant which has been mutated to be protected from proteolytic degradation and thus to be more stable or with mutant Bcl-XL together with XBP-1. Stable cell pools of each genotype are then subjected to seed-stock cultivation and the specific productivity is analysed over several serial passages (FIG. 6).

Similar to the results with XIAP, heterologous expression of Bcl-XL alone has only marginal effects on the productivity of the IgG producer cell line (data not shown). However, the combined expression of XBP-1 and the Bcl-XL mutant again results in a marked increase in the cell's specific productivity. Thus, the combination of XBP-1 and the Bcl-XL mutant yields principally the same results as seen with XBP-1 and XIAP (FIG. 5A).

Taken together, these results demonstrate the applicability of both, XIAP or Bcl-XL to enhance the specific productivity of antibody producer cells in combination with XBP-1. Both proteins are known antagonists of apoptosis, but XIAP acts by inhibiting caspases whereas Bcl-XL exerts its ptotic role by preventing the uncontrolled efflux of apoptogenic molecules from mitochondria. Despite these different modes of action, both proteins are effective in this multigene-engineering approach, suggesting a more general effect which might be broadly applicable for any protein with anti-apoptotic function.

Notably, the extend of enhancement achieved by using the Bcl-XL mutant is not as strong as with XIAP. We also tested the wildtype form of Bcl-XL together with XBP-1 in the same experimental setting, but the increase in the specific antibody productivities was even lower than with the Bcl-XL deletion mutant, which is most likely to be due to higher protein levels of the mutant within the cell as a result of improved protein stability. These results furthermore suggest, that the extend of enhancement depends on the transgene combination and that it will be crucial to identify the most effective pair of secretion enhancing and anti-apoptotic transgenes.

### Example 6

### Multigene-Engineering Using XBP-1 in Combination with Anti-Apoptotic Genes Increases Biopharmaceutical Protein Production of an Antibody

We want to test whether heterologous co-expression of XBP-1 and an anti-apoptotic gene will not only lead to an increase of the specific productivity but in addition to prolonged is cell survival in production processes.

a) To test this, an antibody producing CHO cell line (CHO DG44) secreting humanised anti-CD44v6 IgG antibody BIWA 4 is stably transfected with an empty vector (MOCK control) or expression constructs encoding XBP-1 and XIAP, either from the same or two separate plasmids, or with plasmids carrying XBP-1 and either wild type or mutant Bcl-XL. Subsequently, the newly generated stable cell pools are subjected to batch or fed-batch fermentations. Total cell numbers and cell viabilities are measured daily and at days 3, 5, 7, 9 and 11, samples are taken from the cell culture fluid to determine the IgG titer and the specific productivity.

Within the first days of the production process, both cell growth curves and viabilities of mock and XBP-1/XIAP transfected cells are very similar. However in the later stages when the viability of the control cells starts to decline, XBP-1 and XIAP expressing cells continue to grow at high viabilities over a prolonged time, resulting in a higher IVC at the end of the process. At the same time, cells engineered to express XBP-1 and XIAP together display increase specific productivities. Taken together, this leads to a clear increase in overall product titers in the production process.

b) CHO host cells (CHO DG44) are first transfected with vectors encoding the spliced form of XBP-1 and XIAP or XBP-1 and wildtype or mutant Bcl-XL. Cells are subjected to selection pressure and cell lines are picked that demonstrate heterologous expression of both transgenes. In the case of Bcl-XL expressing cell lines, one or several rounds of gene amplification using the DHFR/MTX- or glutamine-synthetase/MSX-systems are optionally performed. Subsequently, these cell lines and in parallel CHO-DG44 wild type cells are transfected with vectors encoding humanized anti-CD44v6 IgG antibody BIWA 4 as the gene of interest. After a second round of selection, supernatant is taken from seed-stock cultures of all stable cell pools over a period of six subsequent passages, the IgG titer is determined by ELISA and divided by the mean number of cells to calculate the specific productivity. The highest values are seen in the cell pools harbouring XBP-1 and XIAP, followed XBP-1 together with mutant Bcl-XL and XBP-1/Bcl-XL wild type. Importantly, in all cells expressing both XBP-1 and an anti-apoptotic gene, IgG expression is markedly enhanced compared to cells that don't express either or only one of the transgenes.

Very similar results can be obtained if the stable transfectants are subjected to batch or fed-batch fermentations. In each of these settings, combined overexpression of secretion-enhancing and anti-apoptotic gene leads to increased antibody secretion, indicating that by this multi-gene engineering approach, it is possible to enhance cell growth and specific production capacities of the cells in serial cultures or in bioreactor batch or fed batch cultures.

### Example 7

### Overexpression of XBP-1 in Combination with an Anti-Apoptotic Gene Increases Biopharmaceutical Protein Production of Monocyte Chemoattractant Protein 1 (MCP-1)

a) A CHO cell line (CHO DG44) secreting human MCP-1 is stably transfected either with an empty vector (MOCK control) or expression constructs encoding XBP-1 or XIAP or both proteins. The cells are than subjected to selection to obtain stable cell pools. During six subsequent passages, cells are taken from seed-stock cultures of all stable cell pools and the MCP-1 titer is determined by ELISA and the specific productivity is calculated by dividing the titer by the number of viable cells over time.

In XBP-1 transfected cell pools, the specific MCP-1 productivity is markedly higher compared to mock control cells, whereas introduction of XIAP alone has no significant effect. However, the highest MCP-1 titers and specific productivity levels are measured in cells containing both XBP-1 and XIAP.

Next, the same stable cell pools are subjected to batch or fed-batch fermentations. Total cell numbers and cell viabilities are measured daily and at days 3, 5, 7, 9 and 11, samples are taken from the cell culture fluid to determine the MCP-1 titer and the specific productivity.

Within the first days of the production process, cell growth curves and viabilities of mock and XBP-1/XIAP transfected cells are very similar. However in the later stages when the viability of the control cells starts to decline, both XIAP and XBP-1/XIAP expressing cells continue to grow at high viabilities over a prolonged time, resulting in a higher IVC at the end of the process. Furthermore and in agreement with the data obtained in seed stock cultures, XBP-1/XIAP cells display significantly enhanced specific productivities compared to mock and also XBP-1 expressing cells. Taken together, enhanced productivity and prolonged viability result in a clear increase in overall MCP-1 titers in the production process.

b) CHO host cells (CHO DG44) are first transfected with vectors encoding the spliced form of XBP-1 and XIAP, or XBP-1 and BclXL. Cells are subjected to selection pressure to generate stable pools. These are than subjected to single-cell deposition to obtain monoclonal cell lines displaying heterologous expression of both transgenes. In the case of Bcl-XL expressing cell lines, one or several rounds of gene amplification using the DHFR/MTX- or glutamine-synthetase/MSX-systems are optionally performed. Subsequently, these cell lines and in parallel CHO-DG44 wild type cells are transfected with vectors encoding humanized anti-CD44v6 IgG antibody BIWA 4 as the gene of interest. After a second round of selection, supernatant is taken from seed-stock cultures of all stable cell pools over a period of six subsequent passages, the IgG titer is determined by ELISA and divided by the mean number of cells to calculate the specific productivity. The highest values are seen in the cell pools harbouring XBP-1/XIAP, followed XBP-1/Bcl-XL. Importantly, in all cells expressing both XBP-1 and an anti-apoptotic gene, MCP-1 expression is markedly enhanced compared to cells that don't express either or only one of the transgenes.

Very similar results can be obtained if the stable transfectants are subjected to batch or fed-batch fermentations. In each of these settings, combined overexpression of secretion-enhancing and anti-apoptotic gene leads to increased MCP-1 secretion, indicating that by this multi-gene engineering approach, it is possible to enhance cell growth and specific production capacities of the cells in serial cultures or in bioreactor batch or fed batch cultures.

### Example 8

### Overexpression of XBP-1 and XIAP Increases Biopharmaceutical Protein Production of Transmembrane Protein Epithelial Growth Factor Receptor (EGFR)

a) A CHO cell line (CHO DG44) expressing the epithelial growth factor receptor on the cell surface is stably transfected either with an empty vector (MOCK control) or expression constructs encoding XBP-1 or XIAP or both proteins (XBP-1/XIAP). The cells are then subjected to selection to obtain stable cell pools which are subjected to seed stock cultivation. Each week, cell samples are taken from each genotype and the level of EGFR expression is determined by Western Blot or immuno fluorescence staining using specific antibodies.

Cell lines transfected with both, XBP-1 and XIAP display the highest abundance of EGFR on the cell surface. In XBP-1 expressing cells, the signal is also markedly higher compared to control and XIAP expressing cells, but lower than in the double-transgenic cell lines.

The same ranking in cell surface EGFR expression is maintained when the same cells are subjected to batch or fed-batch fermentations and the amount of EGFR on the cells is quantified at different time points during the process.

b) CHO host cells (CHO DG44) are first transfected with vectors encoding the spliced form of XBP-1 and XIAP, or XBP-1 and BclXL. Cells are subjected to selection pressure to generate stable pools. These are than subjected to single-cell deposition to obtain monoclonal cell lines displaying heterologous expression of both transgenes. In the case of Bcl-XL expressing cell lines, one or several rounds of gene amplification using the DHFR/MTX- or glutamine-synthetase/MSX-systems are optionally performed. Subsequently, these cell lines and in parallel CHO-DG44 wild type cells are transfected with vectors encoding the human EGFR as the gene of interest. After a second round of selection, stable EGFR expressing cell pools are obtained from each of the different transgenic host cell lines. When the amount of EGFR protein on the cells is quantified by western blot or immunofluorescence, cells derived from XBP-1/XIAP host cells show the highest EGFR signal compared to controls, followed by XBP-1 expressing cells. These results are independent of the culture format, as the same data are obtained in serial cultures and in batch or fed-batch processes.

In each of these settings, combined overexpression of secretion-enhancing and anti-apoptotic gene leads to an elevated presence of the EGFR on the cell surface, indicating that by this multi-gene engineering approach, it is possible to enhance not only protein secretion but also the abundance of transmembrane proteins on the cell surface.

### Example 9

### Apoptosis Induction in Transiently Transfected Cho-K1 Cells Expressing XBP-1(S)

To analyze whether overexpression of XBP-1 leads to increased apoptosis in cells, CHO-K1 cells are transfected and are analyzed 48 h later by Annexin V assay. Transient transfection is the first step for any cell line generation. Furthermore, transgene levels are highest during this period thereby giving the opportunity to detect a possible apoptosis induction solely by the presence of high XBP-1(s) levels when compared to mock transfected cells. Furthermore, we want to see whether co-expression of the apoptosis-inhibitor protein XIAP is able to reduce apoptosis induction following XBP-1 expression. For this purpose, adherently growing CHO-K1 cells are transfected with either an empty expression plasmid (Mock) or expression constructs encoding XBP-1, XIAP or both proteins (XBP-1/XIAP).

The results of three independent experiments are summarised in FIG. 7. Compared to mock transfected cells, the apoptosis rate is significantly elevated in cells expressing XBP-1 alone, indicating that forced expression of XBP-1 indeed leads to induction or increased sensitivity towards apoptosis. In contrast, apoptosis is clearly reduced in XIAP-transfected cells compared to mock, which demonstrates functional expression of this anti-apoptotic protein. Most importantly, cells expressing both transgenes show lower apoptotic rates than cells expressing solely XBP-1 and even mock cells, thus providing the proof-of-concept that co-introduction of XIAP together with XBP-1 diminishes apoptotic cell death induced by XBP-1 overexpression. This means, that by co-engineering of cells with an anti-apoptotic transgene together with XBP-1, it is possible to overcome XBP-1 induced apoptosis.

### Example 10

### Decreasing XBP-1 Expression and Specific Productivities in Long Term Cultures

If XBP-1 exerts a negative effect on cell growth and survival, this would represent a strong negative selection pressure on XBP-1 expressing cells, which favours every mutation or regulatory mechanism leading to decreased XBP-1 expression. To investigate the long-term stability of heterologous XBP-1(s) expression in the stable CHO cell lines, two cell clones stably expressing XBP-1 (clone E23 and E27) are kept in seed-stock cultures for 35 passages. At passage 10 and passage 35, the abundance of XBP-1(s) mRNA is quantitatively analyzed by real-time PCR. In addition, samples from the cell culture supernatant are taken to also determine the phenotypic stability of the cells in early and late passages in terms of their specific productivity.

As shown in FIG. 8A, XBP-1 transcript levels for both cell clones are higher in the early passage (P10) compared to passage 35. Although the initial expression level in both cell lines (E23 shown in black, E27 in grey) are different, the decrease in XBP-1 expression over time is similar in both cell lines: After 20 passages, XBP-1 expression in both clones has dropped to about 35% of the initial level. This indicates that XBP-1 expression is not stable over time, which might be due to a negative selection pressure disfavoring the synthesis of this transgene.

To test the impact of this loss in heterologous mRNA expression on the specific IgG productivity of the cells, a fed-batch process is performed with both cell lines at the respective passages. The antibody production rate is determined at four time points during the 10 day process and the specific productivity is calculated by dividing the integral of viable cells by the product titer. FIG. 8B shows, that in correlation with the reduction of XBP-1 mRNA, also the mean specific productivity of both cell clones decreases over time. The reduction in productivity is not as pronounced as the drop in XBP-1 mRNA levels, however the trend can be seen in both cell lines (clone E23 in black and clone E27 in grey).

Together, these data indicate that there is a trend towards reducing or silencing XBP-1 expression over time in cells and that this decrease in XBP-1 expression in turn results in a reduction of the specific productivity.

### Example 11

### Increased Expression of XBP-1 in Engineered Cells

To introduce the secretion enhancing gene XBP-1 into antibody producing cell lines, said cells are stably transfected with either a vector backbone alone (“Mock”) or expression constructs encoding XBP-1 or XBP-1 and the anti-apoptotic protein XIAP (XBP-1/XIAP). From the resulting cell populations, total mRNA is prepared and analysed for XBP-1-specific mRNA levels by real-time PCR using beta-tubulin for normalization.

As shown in FIG. 9, cell pools stably transfected to express XBP-1 exhibit markedly higher XBP-1 mRNA levels compared to mock transfected control cells. Moreover, cells expressing the anti-apoptotic protein XIAP show even higher XBP-1 levels, indicating that the presence of XIAP enables the survival of more XBP-1 expressing cells within the population and/or allows even those cells to survive which express XBP-1 at very high levels.

### REFERENCE LIST

Brewer and Hendershot J. (2005). Building an antibody factory: a job for the unfolded protein response. Nat. Immunol. 2005 January; 6(1):23-9.
Campos-da-Paz, M., Costa, C. S., Quilici, L. S., de, C. S., I, Kyaw, C. M., Maranhao, A. Q., and Brigido, M. M. 2008. Production of recombinant human factor VIII in different cell lines and the effect of human XBP1 co-expression. Mol. Biotechnol. 39, 155-158.
Chang B S, Minn A J, Muchmore S W, Fesik S W, Thompson C B. (1997). Identification of a novel regulatory domain in Bcl-X(L) and Bcl-2. Embo J 16(5), 968-977.
Chen-Kiang (2003). Cell-cycle control of plasma cell differentiation and tumorigenesis. Immunol Rev. 2003 August; 194:39-47.
Figueroa, B. Jr. et al. (2001). Biotechnol Bioeng 73, 716-723.
Figueroa, B., Jr., Chen, S., Oyler, G. A., Hardwick, J. M., and Betenbaugh, M. J. (2004). Aven and Bcl-xL enhance protection against apoptosis for mammalian cells exposed to various culture conditions. Biotechnol. Bioeng. 85, 589-600.
Fussenegger, M., Schlatter, S., Datwyler, D., Mazur, X., and Bailey, J. E. (1998). Controlled proliferation by multigene metabolic engineering enhances the productivity of Chinese hamster ovary cells. Nat. Biotechnol. 16, 468-472.
Hengartner, M. O. (2000). The biochemistry of apoptosis. Nature 407, 770-776.
Iwakoshi, N. N., Lee, A. H., and Glimcher, L. H. (2003). The X-box binding protein-1 transcription factor is required for plasma cell differentiation and the unfolded protein response. Immunol Rev 194, 29-38.
Ku, S. C., Ng, D. T., Yap, M. G., and Chao, S. H. (2007). Effects of overexpression of X-box binding protein 1 on recombinant protein production in Chinese hamster ovary and NS0 myeloma cells. Biotechnol. Bioeng., 99, 155-164
Lee, A. H., Chu, G. C., Iwakoshi, N. N., and Glimcher, L. H. (2005). XBP-1 is required for biogenesis of cellular secretory machinery of exocrine glands. EMBO J. 24, 4368-4380.
Ohya, T., Hayashi, T., Kiyama, E., Nishii, H., Miki, H., Kobayashi, K., Honda, K., Omasa, T., and Ohtake, H. 2007. Improved production of recombinant human antithrombin III in Chinese hamster ovary cells by ATF4 overexpression. Biotechnol. Bioeng. 100, 317-324.
Reed, J. C. (2000). Mechanisms of apoptosis. Am. J. Pathol. 157, 1415-1430.
Schroder, M. (2006). The unfolded protein response. Mol. Biotechnol. 34, 279-290.
Shaffer, A. L., Shapiro-Shelef, M., Iwakoshi, N. N., Lee, A. H., Qian, S. B., Zhao, H., Yu, X., Yang, L., Tan, B. K., Rosenwald, A., Hurt, E. M., Petroulakis, E., Sonenberg, N., Yewdell, J. W., Calame, K., Glimcher, L. H., and Staudt, L. M. (2004). XBP1, downstream of Blimp-1, expands the secretory apparatus and other organelles, and increases protein synthesis in plasma cell differentiation. Immunity. 21, 81-93.
Tigges, M. and Fussenegger, M. (2006). Xbp1-based engineering of secretory capacity enhances the productivity of Chinese hamster ovary cells. Metab Eng. 8, 264-272.
Timchenko, N. A., Wilde, M., Nakanishi, M., Smith, J. R., and Darlington, G. J. (1996). CCAAT/enhancer-binding protein alpha (C/EBP alpha) inhibits cell proliferation through the p21 (WAF-1/CIP-1/SDI-1) protein. Genes Dev. 10, 804-815.
Urano, F., Wang, X., Bertolotti, A., Zhang, Y., Chung, P., Harding, H. P., and Ron, D. 2000. Coupling of stress in the ER to activation of JNK protein kinases by transmembrane protein kinase IRE1. Science 287, 664-666.
Urlaub, G., Kas, E., Carothers, A. M., and Chasin, L. A. (1983). Deletion of the diploid is dihydrofolate reductase locus from cultured mammalian cells. Cell 33, 405-412.
Urlaub, G., Mitchell, P. J., Kas, E., Chasin, L. A., Funanage, V. L., Myoda, T. T., and Hamlin, J. (1986). Effect of gamma rays at the dihydrofolate reductase locus: deletions and inversions. Somat. Cell Mol. Genet. 12, 555-566.
Yoshida, H., Haze, K., Yanagi, H., Yura, T., and Mori, K. 1998. Identification of the cis-acting endoplasmic reticulum stress response element responsible for transcriptional induction of mammalian glucose-regulated proteins. Involvement of basic leucine zipper transcription factors. J. Biol. Chem. 273, 33741-33749.
Zinszner, H., Kuroda, M., Wang, X., Batchvarova, N., Lightfoot, R. T., Remotti, H., Stevens, J. L., and Ron, D. 1998. CHOP is implicated in programmed cell death in response to impaired function of the endoplasmic reticulum. Genes Dev. 12, 982-995.

1. A method of producing a heterologous protein of interest in a cell comprising
a. Increasing the expression or activity of a secretion enhancing gene, and
b. Increasing the expression or activity of an anti-apoptotic gene, and
c. Effecting the expression of said protein of interest,
whereby the secretion enhancing gene in step a) is a gene encoding a protein whose expression or activity is induced during one of the following cellular processes: plasma-cell differentiation, unfolded protein response (UPR), endoplasmic reticulum overload response (EOR).

2. The method according to claim 1 whereby
a. The cell has at least 2-fold higher expression levels of the specific mRNA transcript of the secretion enhancing gene in comparison to an untreated control cell and the cell secretes at least 20% more protein-of-interest compared to untransfected cells, and
b. The cell has at least 2-fold higher expression levels of the specific mRNA transcript of the anti-apoptotic-gene in comparison to an untreated control cell.

3. The method according to claim 1 whereby the secretion enhancing gene in step a) is the X-box binding protein-1 (XBP-1) including all XBP-1 splice variants as well as all XBP-1 mutants.

4. The method according to claim 3 whereby the XBP-1 expression level is at least 2-fold higher in comparison to an untreated control cell as measurable by real time PCR using the primers having SEQ ID NOs 17 and 18.

5. The method according to claim 3 whereby the secretion enhancing gene encodes a XBP-1 protein as defined by SEQ ID NO:2.

6. The method according to claim 1 whereby the secretion enhancing gene in step a) is a gene encoding a protein which directly induces the expression or activity of XBP-1.

7. The method according to claim 6 whereby the secretion enhancing gene is IRE, ATF4, ATF6 or IRF4.

8. The method according to claim 1 whereby the secretion enhancing gene in step a) is:
a. a gene whose promoter comprises one or more ER-stress responsive elements (ERSE) as defined by SEQ ID NO:9 or SEQ ID NO:10 or
b. one or more unfolded protein response elements (UPRE) as defined by SEQ ID NO:11 or SEQ ID NO:12, and
whereby said gene is an XBP-1 target gene.

9. The method according to claim 1 whereby the anti-apoptotic gene in step b) is a gene encoding a protein which inhibits or delays the activation of the effector caspases-3 and/or -9.

10. The method according to claim 9 whereby the anti-apoptotic gene is a protein belonging to the inhibitor of apoptosis (IAP) family of proteins which is characterized by one or more copies of an amino acid motive termed BIR (baculovirus IAP repeat) domain.

11. The method according to claim 9 whereby the anti-apoptotic gene comprises a BIR consensus sequence (SEQ ID NO:13).

12. The method according to claim 9 whereby the anti-apoptotic gene is a gene encoding X-linked inhibitor of apoptosis (XIAP) as defined by SEQ ID NO:4.

13. The method according to claim 9 whereby the anti-apoptotic gene is a gene encoding a protein belonging to the Bcl-2 family of proteins which is characterized by its Bcl-2 homology (BH)-domains.

14. The method according to claim 13 whereby the anti-apoptotic gene comprises a Bcl-2 consensus sequence (SEQ ID NO:14).

15. The method according to claim 13 whereby the anti-apoptotic gene is selected from:
a) a gene encoding Bcl-XL (SEQ ID NO:6); and
b) a gene encoding Bcl-XL mutant (SEQ ID NO:8).

16. (canceled)

17. The method according to claim 1 whereby the protein of interest is a membrane or secreted protein.

18. The method according to claim 17 whereby the protein of interest is an antibody or antibody fragment.

19. (canceled)

20. A method of increasing specific cellular productivity of a membrane or secreted protein of interest in a cell comprising introducing into a cell one or more vector systems comprising nucleic acid sequences encoding at least three polypeptides whereby
a. a first polynucleotide encodes a protein having secretion enhancing activity and
b. a second polynucleotide encodes a protein having anti-apoptotic activity and
c. a third polynucleotide encodes a protein of interest and
whereby the protein of interest and the protein having secretion enhancing activity and the protein having anti-apoptotic activity are expressed by said cell and whereby the secretion enhancing gene is a gene encoding a protein whose expression or activity is induced during one of the following cellular processes: plasma-cell differentiation, unfolded protein response (UPR), endoplasmic reticulum overload response (EOR).

21. A method of generating a cell comprising introducing into a cell one or more vector systems comprising nucleic acid sequences encoding at least three polypeptides whereby
a. a first nucleic acid sequence encodes a protein having secretion enhancing activity and
b. a second nucleic acid sequence encodes a protein having anti-apoptotic activity and
c. a third nucleic acid sequence encodes a protein of interest and
whereby the protein of interest and the protein having secretion enhancing activity and the protein having anti-apoptotic activity are expressed by said cell and whereby the secretion enhancing gene is a gene encoding a protein whose expression or activity is induced during one of the following cellular processes: plasma-cell differentiation, unfolded protein response (UPR), endoplasmic reticulum overload response (EOR), and wherein said cell exhibits increased secretion of the protein of interest compared to a cell not comprising the vector systems introduced in steps a and b.

22. The method according to claim 21, whereby the nucleic acid sequence encoding a protein having secretion enhancing activity is XBP-1.

23. The method according to claim 21, whereby the nucleic acid sequence encoding a protein having anti-apoptotic activity is XIAP or a member of the BCL-2 family.

24. A cell generated according to the method of claim 21.

25. The cell according to claim 24 expressing at least three heterologous genes:
a. a secretion enhancing gene,
b. an anti-apoptotic gene, and
c. a protein of interest,
whereby the secretion enhancing gene is XBP 1.

26. (canceled)

27. The cell according to claim 25, whereby the anti-apoptotic gene is XIAP or a member of the BCL-2 family.

28. The cell according to claim 24 whereby said cell is a eukaryotic cell.

29. (canceled)

30. The cell according to claim 28 whereby said eukaryotic cell is a CHO cell selected from CHO wild type, CHO K1, CHO DG44, CHO DUKX-B11, and CHO Pro 5.

31. (canceled)


SEQUENCE LISTING (18 sequences)

SEQ ID NO: 1 - DNA, 1131 bp, Homo sapiens
atggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaa

SEQ ID NO: 2 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 3 - DNA, 1494 bp, Homo sapiens
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaa

SEQ ID NO: 4 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 5 - DNA, 702 bp, Cricetulus griseus
atgtctcagagcaaccgggagctagtggttgactttctctcctacaagctctcccagaaaggatacagctggagtcagtttagtgatgtcgaagagaacaggactgaggccccagaaggaactgaatcagagagggagacccccagtgccatcaatggcaacccatcctggcacctggcggacagccccgcggtaaatggagccactggccacagcagcagtttggatgcacgggaggtgatccccatggcagccgtaaagcaagcgctgagagaggccggcgatgagtttgagctgcggtaccggcgggcgttcagtgatctaacatcccagcttcatataaccccagggactgcatatcaaagctttgaacaggtagtgaatgaactcttccgggatggggtaaactggggtcgcattgtggcctttttctccttcggtggagccctctgtgtggaaagcgtagacaaggagatgcaggtattggtgagtcggatcgcaagttggatggccacctacctgaatgaccacctagagccttggatccaggacaacggcggctgggacactttcgtggaactctacggaaacaatgcagcagctgagagccggaaaggccaggagcgcttcaaccgctggttcctgacgggcatgactgtggctggtgtggttctgctgggctctctcttcagtcggaagtga

SEQ ID NO: 6 - protein, 233 aa, Cricetulus griseus
MSQSNRELVVDFLSYKLSQKGYSWSQFSDVEENRTEAPEGTESERETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIASWMATYLNDHLEPWIQDNGGWDTFVELYGNNAAAESRKGQERFNRWFLTGMTVAGVVLLGSLFSRK

SEQ ID NO: 7 - DNA, 600 bp, Cricetulus griseus
atgtctcagagcaaccgggagctagtggttgactttctctcctacaagctctcccagaaaggatacagctggagtcagtttagtgatgtcgaagagaacaggactgaggccccagaaggaactgaatcagagagggcggccgcagcagcagccgtaaagcaagcgctgagagaggccggcgatgagtttgagctgcggtaccggcgggcgttcagtgatctaacatcccagcttcatataaccccagggactgcatatcaaagctttgaacaggtagtgaatgaactcttccgggatggggtaaactggggtcgcattgtggcctttttctccttcggtggagccctctgtgtggaaagcgtagacaaggagatgcaggtattggtgagtcggatcgcaagttggatggccacctacctgaatgaccacctagagccttggatccaggacaacggcggctgggacactttcgtggaactctacggaaacaatgcagcagctgagagccggaaaggccaggagcgcttcaaccgctggttcctgacgggcatgactgtggctggtgtggttctgctgggctctctcttcagtcggaagtga

SEQ ID NO: 8 - protein, 199 aa, Cricetulus griseus
MSQSNRELVVDFLSYKLSQKGYSWSQFSDVEENRTEAPEGTESERAAAAAAVKQALREAGDEFELRYRRAFSDLTSQLHITPGTAYQSFEQVVNELFRDGVNWGRIVAFFSFGGALCVESVDKEMQVLVSRIASWMATYLNDHLEPWIQDNGGWDTFVELYGNNAAAESRKGQERFNRWFLTGMTVAGVVLLGSLFSRK

SEQ ID NO: 9 - DNA, 19 bp, Homo sapiens
  misc_feature (6)..(14): n is a, c, g, or t
ccaatnnnnnnnnnccacg

SEQ ID NO: 10 - DNA, 11 bp, Homo sapiens
  misc_feature (6)..(6): n is a, c, g, or t
attggnccacg

SEQ ID NO: 11 - DNA, 8 bp, Homo sapiens
tgacgtgg

SEQ ID NO: 12 - DNA, 8 bp, Homo sapiens
tgacgtga

SEQ ID NO: 13 - protein, 66 aa, Artificial
  BIR domain consensus sequence
RLRTFQNWPISNLQFPEQLAKAGFYYTGVGDEVRCFFCGVELKNWEPGDDPWEEHKRWSPNCPFVR

SEQ ID NO: 14 - protein, 99 aa, Artificial
  Bcl-2 domain consensus sequence
LRRAGDELEKRYERAFSSMLVQLHITPETARELFTQVAGELFEDGINWGRIVALFSFGGALAKKLVNAEMEGLVSRLADWMVEFLKHNLAEWIQQNGGW

SEQ ID NO: 15 - DNA, 21 bp, Artificial
  primer Tub_for
ctcaacgccgacctgcgcaag

SEQ ID NO: 16 - DNA, 20 bp, Artificial
  primer Tub_rev
actcgctggtgtaccagtgc

SEQ ID NO: 17 - DNA, 20 bp, Artificial
  primer XBP1_for
tggttgagaaccaggagtta

SEQ ID NO: 18 - DNA, 19 bp, Artificial
  primer XBP1_rev
gcttccagcttggctgatg